(19) United States
(12) Patent Application Publication
(10) Pub. No.: US 2019/0216557 A1
(43) Pub. Date: Jul. 18, 2019

(54) TREATMENT TOOL UNIT, MEDICAL TREATMENT TOOL, AND SURGICAL SYSTEM

(71) Applicant: MEDICAROID CORPORATION, Kobe-shi (JP)

(72) Inventors: Kazuki ISHIHARA, Kobe-shi (JP); Tetsuya NAKANISHI, Kobe-shi (JP); Yasuhiko HASHIMOTO, Kobe-shi (JP); Yu USUKI, Kobe-shi (JP)

(73) Assignee: MEDICAROID CORPORATION, Kobe-shi (JP)

(21) Appl. No.: 16/365,692

(22) Filed: Mar. 27, 2019

Related U.S. Application Data
(63) Continuation of application No. PCT/JP2018/002556, filed on Jan. 26, 2018.

(30) Foreign Application Priority Data
May 1, 2017 (JP) 2017-091233

(51) Int. Cl. A61B 34/30; A61B 17/29; A61B 90/50; A61B 90/00; A61B 34/00

(52) U.S. Cl. CPC A61B 34/30; A61B 17/2909; A61B 90/50; A61B 46/10; A61B 34/71; A61B 2034/305; A61B 90/361

(57) ABSTRACT

A treatment tool unit according to one or more embodiments may be attachable to an actuation unit including an actuation-side shaft which accommodates rigid actuation-side elongate elements. The treatment tool unit may include: a distal end portion; flexible elongate elements coupled to the distal end portion to manipulate the distal end portion; rigid distal end-side elongate elements each including a first end coupled to a corresponding one of the flexible elongate elements and a second end capable of being coupled to a corresponding one of the actuation-side elongate elements; a distal end-side shaft which accommodates the rigid distal end-side elongate elements and is attachable to the actuation-side shaft; and a distal end-side movement restricting portion which restricts movements of the distal end-side elongate elements with respect to the distal end-side shaft toward the distal end portion in an axial direction of the distal end-side shaft.

### CROSS-REFERENCE TO RELATED APPLICATION

This is a continuation of International Application No. PCT/JP2018/002556 filed on Jan. 26, 2018, which claims priority to Japanese Patent Application No. 2017-091233 filed on May 1, 2017. The entire contents of these applications are incorporated herein by reference.

### TECHNICAL FIELD

One or more embodiments relate to a treatment tool unit including a distal end portion such as grasping forceps used in a surgery, a medical treatment tool including the treatment tool unit, and a surgical system.

### BACKGROUND ART

A surgery assisting robot has sometimes been used in the field of endoscopic surgery. The surgery assisting robot includes, for example, a patient-side apparatus including a manipulator. An appropriate medical treatment tool is attached to the manipulator. The medical treatment tool is remotely controlled to carry out surgery.

As an example of such a medical treatment tool, WO 2000/33755 (Patent Document 1), for example, discloses a surgical instrument which includes a distal end portion, a shaft which accommodates an elongate element used to manipulate the distal end portion, and an interface which actuates the elongate element. The surgical instrument is attachable to and detachable from a robot arm.

In addition, Japanese Translation of PCT International Application No. 2015-521905 (Patent Document 2), for example, discloses a surgical device, a distal end portion of which is detachable from a shaft.

### SUMMARY

However, according to the surgical instrument described in Patent Document 1,in order to replace the distal end portion with another, the entire surgical instrument, including the distal end portion, the shaft, and the interface, need to be detached from the robot arm for the replacement, which results in a cost increase.

In addition, according to the surgical device described in Patent Document 2, jaws at the distal end portion are opened and closed by the actuation of a drive screw, which is a rigid elongate element, and the degree of freedom in operation is thus insufficient.

An object of one or more embodiments may be to provide a treatment tool unit, a medical treatment tool, and a surgical system, in which a distal end portion of the treatment tool unit can be manipulated in multiple degrees of freedom and is replaceable without replacing the entire medical treatment tool.

A treatment tool unit of one or more embodiments may be attachable to an actuation unit including an actuation-side shaft which accommodates rigid actuation-side elongate elements. The treatment tool unit may include: a distal end portion; flexible elongate elements coupled to the distal end portion to manipulate the distal end portion; rigid distal end-side elongate elements each including a first end coupled to a corresponding one of the flexible elongate elements and a second end capable of being coupled to a corresponding one of the actuation-side elongate elements; a distal end-side shaft which accommodates the rigid distal end-side elongate elements and is attachable to the actuation-side shaft; and a distal end-side movement restricting portion which restricts movements of the distal end-side elongate elements with respect to the distal end-side shaft toward the distal end portion in an axial direction of the distal end-side shaft when the treatment tool unit is attached to the actuation unit.

### BRIEF DESCRIPTION OF THE DRAWINGS

FIG. 1 is a diagram illustrating a configuration of a surgical system.

FIG. 2 is a diagram illustrating a configuration of a medical treatment tool.

FIG. 3 is a diagram illustrating a perspective view of a configuration of a distal end portion of the medical treatment tool in FIG. 2.

FIG. 4 is a diagram illustrating a side view of the configuration of the distal end portion of the medical treatment tool in FIG. 3.

FIG. 5 is a diagram illustrating a configuration of a combination of a rod and a wire as an example of an elongate element used for a medical treatment tool.

FIG. 6 is a diagram illustrating an example wire-wrapping.

FIG. 7 is a diagram illustrating a configuration of a distal end portion according to a variation of the medical treatment tool.

FIG. 8 is a diagram illustrating a perspective view of configurations of an actuation mechanism and an actuation device.

FIG. 9 is a diagram illustrating a front view of the configuration of the actuation mechanism in FIG. 8.

FIG. 10 is a diagram illustrating a perspective view of a configuration of a variation of the actuation mechanism of one or more embodiments.

FIG. 11 is a diagram illustrating a perspective view of a configuration of a transmitting portion which transmits power between the actuation mechanism and the actuation device illustrated in FIG. 8.

FIG. 12 is a diagram illustrating a perspective view of a configuration of a transmitting portion which transmits power between the actuation mechanism and the actuation device illustrated in FIG. 10.

FIG. 13 is a diagram illustrating a cross-sectional view of a configuration (Example 1) of an attachable/detachable mechanism of the medical treatment tool.

FIG. 14 is a diagram illustrating a cross-sectional view of a configuration (Example 2) of an attachable/detachable mechanism of the medical treatment tool.

FIG. 15 is a diagram illustrating a perspective view of a configuration (Example 3) of an attachable/detachable mechanism of the medical treatment tool.

FIG. 16 is a diagram illustrating a perspective view of a variation of the configuration (Example 3) of the attachable/detachable mechanism of the medical treatment tool.

FIG. 17 is a diagram illustrating a perspective view of an external configuration (Example 4) of an attachable/detachable mechanism of the medical treatment tool.

FIG. 18 is a diagram illustrating a perspective view of an internal configuration of the medical treatment tool in FIG. 17.

FIG. 19 is a diagram illustrating a front view of the medical treatment tool in FIG. 17.

FIG. 20 is a diagram illustrating a cross-sectional view taken along the line XX-XX of FIG. 19.

FIG. 21 is a diagram illustrating a perspective view of a state in which a distal end-side rod and an actuation-side rod, which are shown in FIG. 17, are about to be coupled to each other.

FIG. 22 is a diagram illustrating cross-sectional views taken along the line XXII-XXII of FIG. 20.

FIG. 23 is a diagram illustrating a perspective view of a configuration (Example 5) of an attachable/detachable mechanism of the medical treatment tool.

FIG. 24 is a diagram illustrating a front view of the medical treatment tool in FIG. 23.

FIG. 25 is a diagram illustrating a cross-sectional view taken along the line XXV-XXV of FIG. 24.

### DETAILED DESCRIPTION

Descriptions are provided hereinbelow for embodiments based on the drawings. In the respective drawings referenced herein, the same constituents are designated by the same reference numerals and duplicate explanation concerning the same constituents is omitted. All of the drawings are provided to illustrate the respective examples only.

### First Embodiment

### [Surgical System]

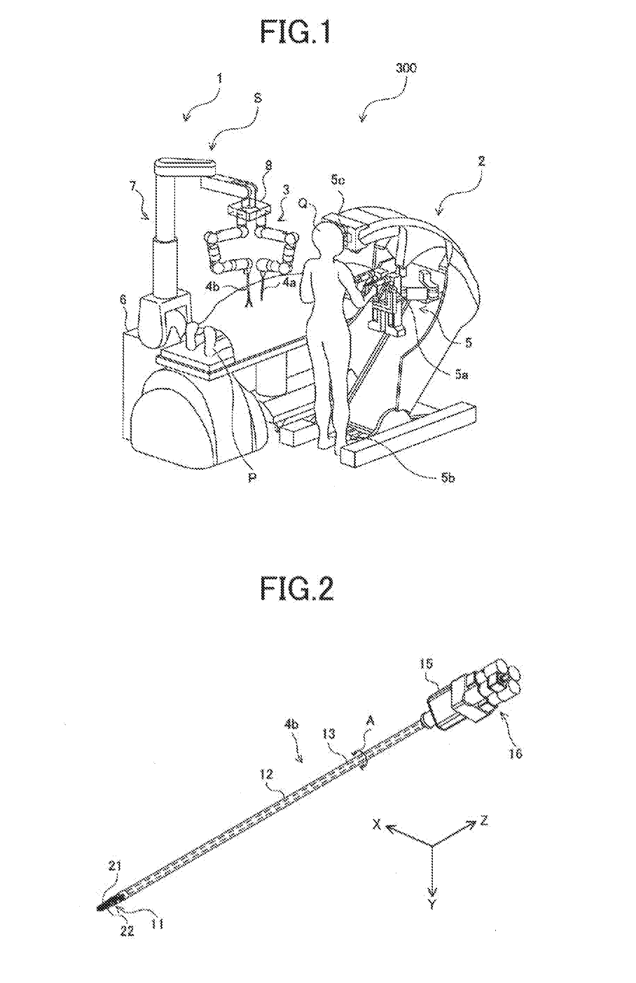

FIG. 1 is a diagram illustrating a configuration of a surgical system. Referring to FIG. 1, a surgical system 300 is used, for example, to carry out a surgical operation on a treatment target P, such as a human or an animal, using an endoscope operated by an operator Q through a patient-side apparatus 1. The surgical system 300 includes the patient-side apparatus 1 and an operating apparatus 2 which operates the patient-side apparatus 1.

The operator Q inputs, to the operating apparatus 2, a movement instruction for the patient-side apparatus 1. The operating apparatus 2 transmits an instruction signal which includes this movement instruction to the patient-side apparatus 1. The patient-side apparatus 1 receives the instruction signal transmitted from the operating apparatus 2, and moves an endoscope assembly 4a and a medical treatment tool 4b connected to the distal end of the patient-side apparatus 1, based on the movement instruction included in the instruction signal received.

More specifically, the operating apparatus 2 includes an operation input section 5 which has a control manipulator 5a and an operation pedal 5b, and a monitor 5c which displays an image taken by the endoscope assembly 4a. The control manipulator 5a and the operation pedal 5b are equipment through which the operator Q inputs the movement instruction.

The operator Q operates the control manipulator 5a and the operation pedal 5b to input the movement instruction to the operating apparatus 2, while viewing an image of the target site displayed on the monitor 5c. The operating apparatus 2 transmits the instruction signal, which includes the movement instruction which has been input, to the patient-side apparatus 1 through a wired or wireless connection.

The patient-side apparatus 1 includes: a positioner 7; a platform 8 attached to an end of the positioner 7; a plurality of manipulators 3 detachably attached to the platform 8; the endoscope assembly 4a; the medical treatment tool 4b; and a controller 6 which controls the movement of the patient-side apparatus 1.

The endoscope assembly 4a and the medical treatment tool 4b are attached to the manipulators 3. Examples of the medical treatment tool 4b include a grasping forceps (i.e., a grasper), a needle holder (i.e., a needle driver), and a pair of scissors.

The controller 6 receives the instruction signal transmitted from the operating apparatus 2, and moves the endoscope assembly 4a and the medical treatment tool 4b, based on the instruction signal received.

Specifically, the controller 6 which has received the instruction signal first moves the positioner 7, thereby positioning the platform 8, based on the movement instruction included in the instruction signal. The controller 6 positions the manipulators 3, too, such that the endoscope assembly 4a and the medical treatment tool 4b take predetermined initial positions with respect to cannulas, not shown, retained on the body surface of the treatment target P.

Then, the controller 6 outputs, based on the movement instruction, a control signal for actuating the endoscope assembly 4a and the medical treatment tool 4b to the manipulators 3. The endoscope assembly 4a and the medical treatment tool 4b move according to the control signal transmitted from the controller 6 and received in the manipulators 3. Movements of the endoscope assembly 4a and the medical treatment tool 4b are controlled in this manner.

### [Medical Treatment Tool]

(General Configuration)

FIG. 2 is a diagram illustrating a configuration of a medical treatment tool.

Referring to FIG. 2, the medical treatment tool 4b includes a distal end portion 11, a shaft 12, a plurality of elongate elements 13 for operating the distal end portion 11, and an actuation mechanism 15 for actuating the elongate elements 13. The actuation mechanism 15 is actuated by an actuation device 16 having a driving source. The actuation device 16 is, for example, a part of the manipulator 3 and is provided at a distal end of the manipulator 3.

In this specification, the term “elongate element” is used as a general term for a rod, a wire, a cable, or the like. In particular, a rigid material such as a rod is referred to as a “rigid elongate element” and a flexible material such as a wire or a cable is referred to as a “flexible elongate element.” In a case in which these terms are not distinguished from each other, both rigid and flexible elongate elements are collectively referred to as an “elongate element.”

The distal end portion 11 has two jaws 21 and 22, for example. The two jaws 21 and 22 having the same shape can reduce the fabrication costs. The shaft 12 has a tubular shape extending in the longitudinal direction of the medical treatment tool 4b, and is rotatable in the directions of arrows A. That is, the shaft 12 is rotatable about its own longitudinal axis.

A rod as an example of the elongate element 13 is a rigid member accommodated in the shaft 12. A wire as an example of the elongate element 13 is a flexible member made of tungsten, stainless steel, or the like and having sufficient strength, bendability, and durability.

The actuation mechanism 15 is detachably coupled to the manipulator 3 of the patient-side apparatus 1 illustrated in FIG. 1. The actuation device 16 receives the control signal from the controller 6 and is actuated by this control signal, which causes the actuation mechanism 15 to move the elongate element 13 along the longitudinal direction of the medical treatment tool 4b. Detailed configurations of the actuation mechanism 15 will be described later.

(Distal End Portion)

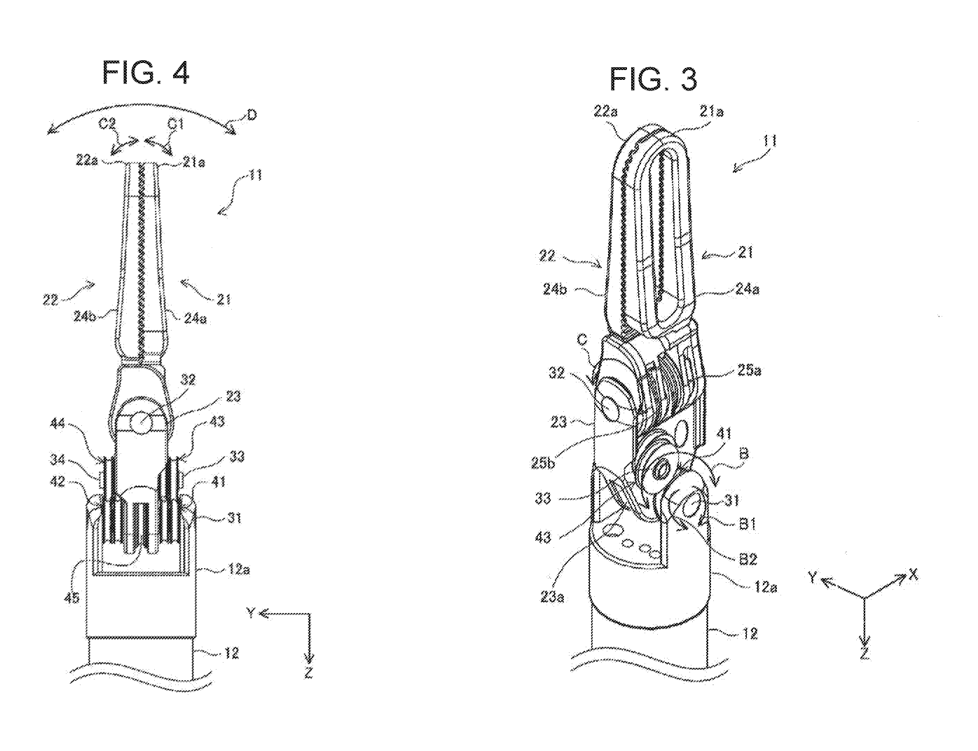

FIG. 3 is a diagram illustrating a perspective view of a configuration of the distal end portion of the medical treatment tool in FIG. 2.

Referring to FIG. 3, the distal end portion 11 also has a wrist portion 23 in addition to the jaws 21 and 22. The wrist portion 23 is mounted on an end 12a of the shaft 12 via a connection 31. The wrist portion 23 is pivotable about the connection 31 in the directions of arrows B.

The jaws 21 and 22 are mounted on the wrist portion 23 via a connection 32. The jaws 21 and 22 have finger portions 24a and 24b and pulley portions 25a and 25b, respectively. The finger portions 24a and 24b are pivotable about the connection 32 in the directions of arrows C. The pulley portions 25a and 25b are rotatable about the connection 32.

In a preferred embodiment, the connection 31 and the connection 32 extend in different directions. In the present embodiment, the direction in which the connection 31 extends and the direction in which the connection 32 extends form a 90 degree angle. In the following description, the direction in which the connection 31 extends will be referred to as a “Y-axis direction,” the direction in which the connection 32 extends as an “X-axis direction,” and the longitudinal direction of the shaft 12 as a “Z-axis direction.”

FIG. 4 is a diagram illustrating a side view of the configuration of the distal end portion of the medical treatment tool in FIG. 3.

Referring to FIG. 4, the jaws 21 and 22 have free ends 21a and 22a, respectively. The free ends 21a and 22a pivot about the connection 32, and thus can move toward and away from each other or can pivot in the same direction, as indicated by the arrows C1 and C2.

The distal end portion 11 also has a first pulley portion 41, a second pulley portion 42, a third pulley portion 43, a fourth pulley portion 44, and a fifth pulley portion 45 in addition to the jaws 21 and 22 and the wrist portion 23. Each of the first, second, third, and fourth pulley portions 41, 42, 43, and 44 has an inner pulley and an outer pulley.

The first, second, and fifth pulley portions 41, 42, and 45 are mounted on the end 12a via the connection 31, and are rotatable about the connection 31. The third pulley portion 43 is mounted on the wrist portion 23 via a connection 33, and is rotatable about the connection 33. The fourth pulley portion 44 is mounted on the wrist portion 23 via a connection 34, and is rotatable about the connection 34.

The plane of rotation of the first pulley portion 41 and the plane of rotation of the third pulley portion 43 are present on substantially the same plane. The plane of rotation of the second pulley portion 42 and the plane of rotation of the fourth pulley portion 44 are present on substantially the same plane.

(Rod and Wire)

FIG. 5 is a diagram illustrating a configuration of a combination of a rod and a wire as an example of an elongate element used for a medical treatment tool.

Referring to FIG. 5, the elongate element 13 includes a first rod (a rigid actuation-side elongate element) 13a, a second rod (a rigid actuation-side elongate element) 13b, and a wire (a flexible elongate element) 14. The first rod 13a and the second rod 13b are connected to the wire 14. The wire 14 has a thick portion 14a at around a middle portion of the wire.

In this example, the first rod 13a and the second rod 13b are connected to the same wire 14. Note that the wire 14 to which the first rod 13a is connected and the wire 14 to which the second rod 13b is connected may be different members.

The first rod 13a and the second rod 13b have grooves 20a and 20b, respectively, at the ends opposite to the ends toward the wire 14. The grooves 20a and 20b engage with gears of the actuation mechanism 15

The medical treatment tool 4b shown in FIG. 2 includes, for example, three sets of the first rod 13a, the second rod 13b, and the wire 14. Hereinafter, the first rods 13a included in the respective three sets will be referred to as the first rods 131a, 132a, and 133a. Likewise, the second rods 13b included in the respective three sets will be referred to as the second rods 131b, 132b, and 133b. The three wires 14 included in the three sets will be referred to as wires 141, 142, and 143.

The wire 141 connects the first rod 131a and the second rod 131b. The wire 142 connects the first rod 132a and the second rod 132b. The wire 143 connects the first rod 133a and the second rod 133b.

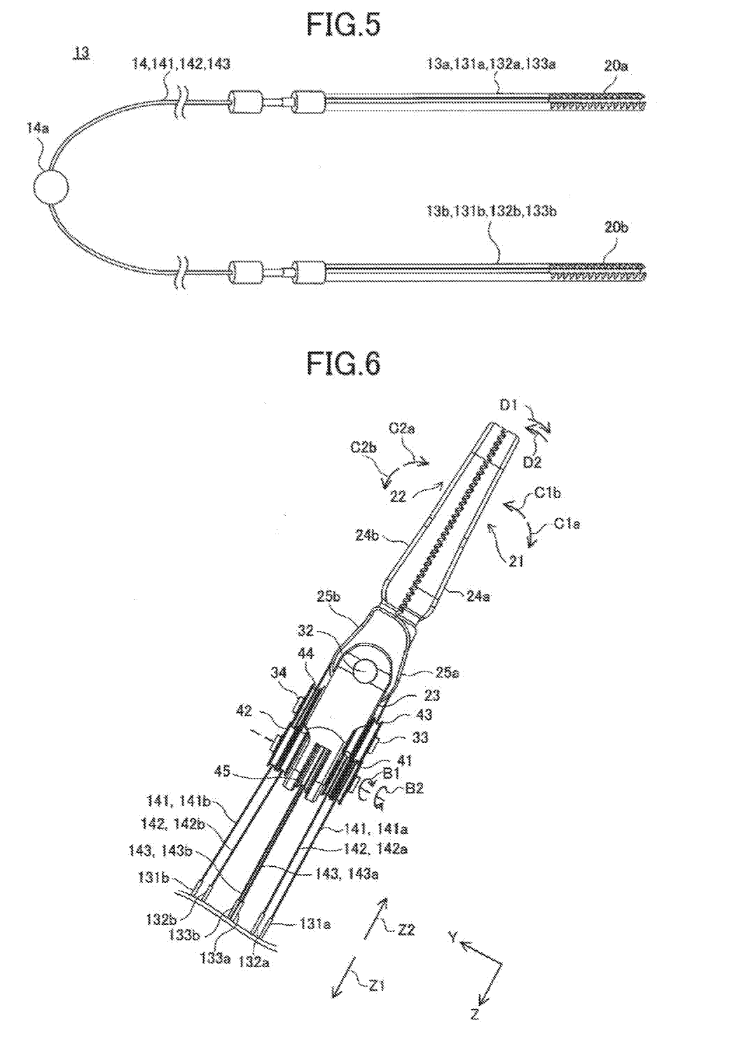

FIG. 6 is a diagram illustrating an example wire-wrapping. In FIG. 6, the arrow Z1 indicates the positive direction of the Z-axis, and the arrow Z2 indicates the negative direction of the Z-axis.

Referring to FIG. 6, the medical treatment tool 4b is assembled as follows: the wire 141 is wrapped around the outer pulley of the first pulley portion 41 and the outer pulley of the third pulley portion 43; the wire 141 is then wrapped around the pulley portion 25b of the jaw 22; and the wire 141 is further wrapped around the inner pulley of the fourth pulley portion 44 and the inner pulley of the second pulley portion 42.

The thick portion 14a of the wire 141 shown in FIG. 5 is fitted, for example, into a recess (not shown) formed in the finger portion 24b of the jaw 22 shown in FIG. 6. The wire 141 is fixed to the jaw 22 in this manner. The jaw 22 therefore moves in conjunction with the movement of the wire 141.

The wire 142 is wrapped around the inner pulley of the first pulley portion 41 and the inner pulley of the third pulley portion 43; the wire 142 is then wrapped around the pulley portion 25a of the jaw 22; and the wire 142 is further wrapped around the outer pulley of the fourth pulley portion 44 and the outer pulley of the second pulley portion 42.

The thick portion 14a of the wire 142 shown in FIG. 5 is fitted, for example, into a recess (not shown) formed in the finger portion 24a of the jaw 21 shown in FIG. 6. The wire 142 is fixed to the jaw 21 in this manner. The jaw 21 therefore moves in conjunction with the movement of the wire 142.

The wire 143 is wrapped around the fifth pulley portion 45. The thick portion 14a of the wire 143 shown in FIG. 5 is fitted, for example, into a recess (not shown) formed in the wrist portion 23 shown in FIG. 6. The wire 143 is fixed to the wrist portion 23 in this manner. The wrist portion 23 therefore moves in conjunction with the movement of the wire 143.

In this manner, the jaws 22 and 21 and the wrist portion 23 provided at the distal end portion 11 are configured to move in conjunction with the movements of the wire 141, 142, and 143, respectively, which are flexible members. This configuration allows the distal end portion 11 to move smoothly in a desired direction.

(Movement of Distal End Portion)

The first rod 131a, when pulled in the Z1 direction, causes the jaw 22 to pivot about the connection 32 in the direction of arrow C2a, that is, to pivot circumferentially about the connection 32 toward the jaw 21. Similarly, the second rod 131b, when pulled in the Z1 direction, causes the jaw 22 to pivot about the connection 32 in the direction of arrow C2b, that is, to pivot circumferentially about the connection 32 away from the jaw 21.

The first rod 132a, when pulled in the Z1 direction, causes the jaw 21 to pivot about the connection 32 in the direction of arrow C1a, that is, to pivot circumferentially about the connection 32 away from the jaw 22. The second rod 132b, when pulled in the Z1 direction, causes the jaw 21 to pivot about the connection 32 in the direction of arrow C1b, that is, to pivot circumferentially about the connection 32 toward the jaw 22.

The second rod 131b and the first rod 132a, when simultaneously pulled in the Z1 direction, cause the jaws 21 and 22 to pivot circumferentially about the connection 32 away from each other. Similarly, the first rod 131a and the second rod 132b, when simultaneously pulled in the Z1 direction, cause the jaws 21 and 22 to pivot circumferentially about the connection 32 toward each other.

The first rod 131a and the first rod 132a, when simultaneously pulled in the Z1 direction, cause both of the jaws 21 and 22 to pivot circumferentially about the connection 32 in the direction of arrow D1. That is, the jaw 21 pivots in the C1a direction, and the jaw 22 pivots in the C2a direction.

The second rod 131b and the second rod 132b, when simultaneously pulled in the Z1 direction, cause both of the jaws 21 and 22 to pivot circumferentially about the connection 32 in the direction of arrow D2. That is, the jaw 21 pivots in the C1b direction, and the jaw 22 pivots in the C2b direction.

The first rod 133a, when pulled in the Z1 direction, causes the wrist portion 23 illustrated in FIGS. 3 and 4 to pivot about the connection 31 in a direction of arrow B2, that is, to pivot circumferentially about the connection 31 and counterclockwise as viewed from the negative to positive direction of the Y-axis. Likewise, the second rod 133b, when pulled in the Z1 direction, causes the wrist portion 23 to pivot about the connection 31 in a direction indicated by the arrow B1, that is, to pivot circumferentially about the connection 31 and clockwise as viewed from the negative to positive direction of the Y-axis.

In this manner, the jaw 22, the jaw 21, and the wrist portion 23, which are respectively fixed to the wires 141, 142, and 143, move independently of one another in conjunction with the movements of the first rods 131a, 132a, and 133a and the second rods 131b, 132b, and 133b. That is, the distal end portion 11 can be manipulated in multiple degrees of freedom.

### [Variation of Medical Treatment Tool]

The medical treatment tool 4b according to one or more embodiments is characterized in that an attachable/detachable mechanism, which will be described later, enables a treatment tool unit including the distal end portion 11 to be detached from, and attached to, the shaft 12 even in a case where the distal end portion 11 is manipulated by a flexible elongate element (e.g., the wire 14) connected to the distal end portion 11. However, it is not intended to exclude a case in which the attachable/detachable mechanism is applied to a medical treatment tool 4b having a distal end portion 11 manipulated by a rigid elongate element (e.g., a rod) connected to the distal end portion 11.

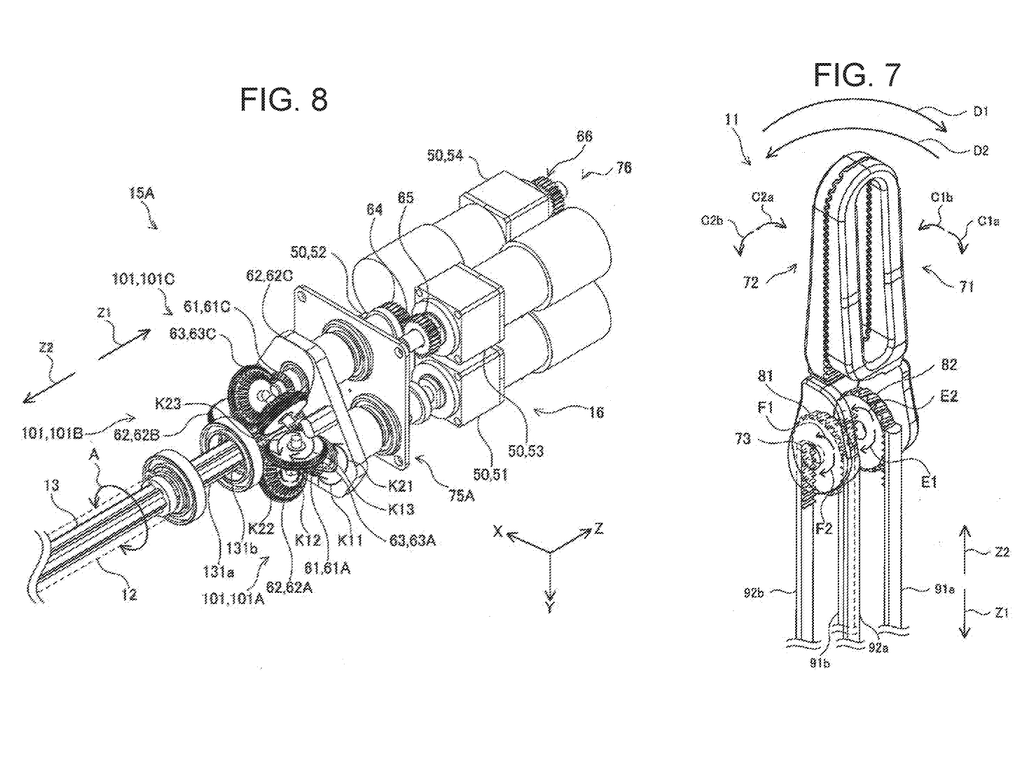

FIG. 7 is a diagram illustrating a configuration of a distal end portion according to a variation of the medical treatment tool.

Referring to FIG. 7, the distal end portion 11 has two jaws 71 and 72 and two gears 81 and 82, for example. The two jaws 71 and 72 and the two gears 81 and 82 are mounted on the end 12a of the shaft 12, shown in FIG. 3, via a connection 73. The gears 81 and 82 are rotatable about the connection 73, and the jaws 71 and 72 are pivotable about the connection 73 in the direction of arrow D1 or D2.

The medical treatment tool 4b also includes first rods 91a and 92a and second rods 91b and 92b, instead of the plurality of rods 131a, 131b, 132a, 132b, 133a, and 133b, and the plurality of wires 141, 142, and 143.

The first rod 91a and the second rod 91b are arranged such that grooves formed at an end of each rod face each other with the gear 82 interposed between the grooves of the rods. Movements of the first rod 91a and the second rod 91b in opposite directions from each other in the Z-axis direction cause the gear 82 to rotate about the connection 73, and the jaw 71 to rotate about the connection 73 together with the gear 82.

Similarly, the first rod 92a and the second rod 92b are arranged such that grooves formed at an end of each rod face each other with the gear 81 interposed between the grooves of the rods. Movements of the first rod 92a and the second rod 92b in opposite directions from each other in the Z-axis direction cause the gear 81 to rotate about the connection 73, and the jaw 72 to rotate about the connection 73 together with the gear 81.

Specifically, the first rod 91a, when pulled in the Z1 direction, causes the second rod 91b to move in the Z2 direction, the gear 82 to rotate in the E1 direction, and the jaw 71 to pivot in the C1a direction. Likewise, the first rod 91a, when pulled in the Z2 direction, causes the second rod 91b to move in the Z1 direction, the gear 82 to rotate in the E2 direction, and the jaw 71 to pivot in the C1b direction.

The first rod 92a, when pulled in the Z1 direction, causes the second rod 92bto move in the Z2 direction, the gear 81 to rotate in the F2 direction, and the jaw 72 to pivot in the C2a direction. The first rod 92a, when pulled in the Z2 direction, causes the second rod 92b to move in the Z1 direction, the gear 81 to rotate in the F1 direction, and the jaw 72 to pivot in the C2b direction.

In such a case as this, in which the first rods 91a and 92a and the second rods 91b and 92b are coupled directly to the distal end portion 11, a step of, e.g., adjusting the wire tension is not necessary in a process of, e.g., assembling the medical treatment tool 4b. The above-disclosed configuration may further simplify the process of, e.g., assembling the medical treatment tool 4b.

### [Actuation Mechanism and Actuation Device]

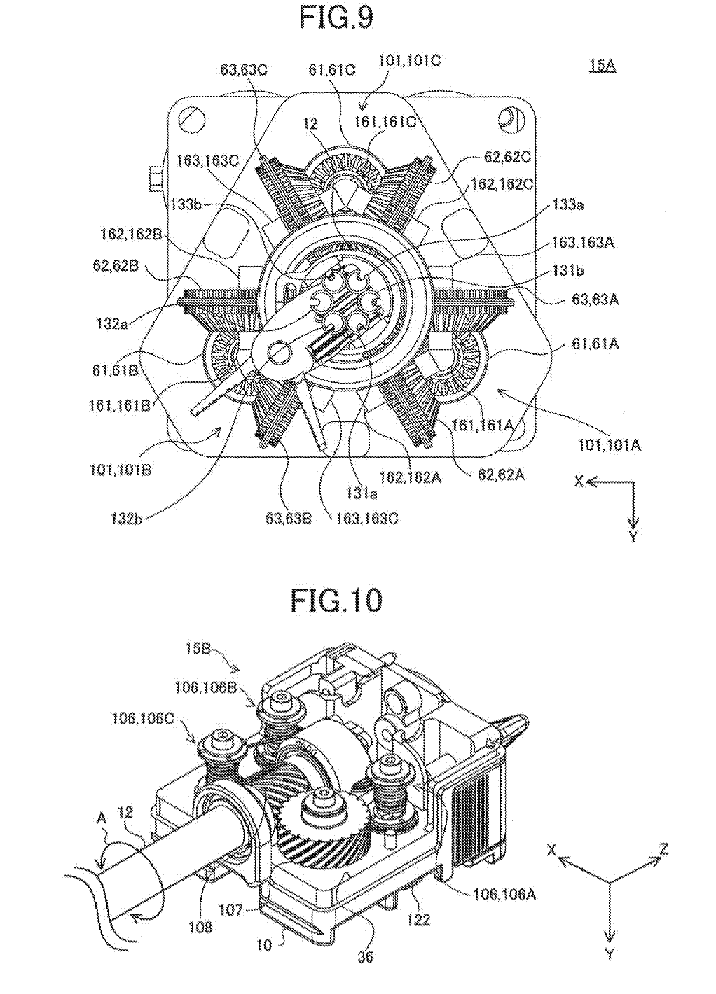

FIG. 8 is a diagram illustrating a perspective view of configurations of an actuation mechanism and an actuation device. FIG. 9 is a diagram illustrating a front view of the configuration of the actuation mechanism in FIG. 8. In FIGS. 8 and 9, the housing which covers the actuation mechanism 15A is omitted to illustrate the actuation mechanism 15A.

Referring to FIG. 8, the actuation mechanism 15A includes a plurality of actuation units 101. The actuation unit 101 includes a first gear 61, a second gear 62 engaged with the first rod 13a, a third gear 63 engaged with the second rod 13b, and a receiving member which is a part of a transmitting portion 75A for rotating the first gear 61. Details of the transmitting portion 75A will be described later.

The actuation device 16 has a plurality of actuators 50. Each of the actuators 50 receives the control signal from the patient-side apparatus 1 illustrated in FIG. 1 via the manipulator 3. Based on this control signal, the actuator 50 rotates the receiving member of the transmitting portion 75A. The rotation of the receiving member causes the first gear 61, associated with the receiving member, to rotate.

More particularly, referring to FIG. 9, the second gear 62 and the third gear 63 have substantially the same shape. The first gear 61 rotates about a first axis 161 which is parallel to the longitudinal direction of the medical treatment tool 4b. The second gear 62 rotates about a second axis 162 extending in a direction perpendicular to the first axis 161. The third gear 63 rotates about a third axis 163 extending in a direction perpendicular to the first axis 161.

The first gear 61 is, for example, a substantially conical bevel gear, a conical surface of which is provided with a groove. The second gear 62 and the third gear 63 are, for example, a combination of a substantially conical bevel gear and a cylindrical gear, wherein a groove is formed in a conical surface of the conical portion and a groove is formed in a side surface of the cylindrical portion.

The second gear 62 and the third gear 63 engage with the first gear 61. More particularly, the groove formed in the conical surface of the second gear 62 and the groove formed in the conical surface of the third gear 63 engage with the groove formed in the first gear 61.

In conjunction with the rotation of the first gear 61, the second gear 62 and the third gear 63 rotate in the same direction as viewed from the first gear 61. That is, when the second gear 62 rotates clockwise as viewed from the first gear 61, the third gear 63 also rotates clockwise as viewed from the first gear 61.

In the above example, all of the first, second, and third gears 61, 62, and 63 are comprised of bevel gears. In another example, the first gear 61 may be comprised of a crown gear, and the second and third gears 62 and 63 may be comprised of spur gears. In this case, as well, the same rotation operation as the rotation operation described above can be realized.

The groove formed in the side surface of the cylindrical portion of the second gear 62 engages with the groove 20a formed in the first rod 131a illustrated in FIG. 5. The second gear 62 and the first rod 131a constitute a rack and pinion structure. The first rod 131a moves along the longitudinal direction of the shaft 12 in conjunction with the rotation of the second gear 62.

The groove formed in the side surface of the cylindrical portion of the third gear 63 engages with the groove 20b formed in the second rod 131b illustrated in FIG. 5. The third gear 63 and the second rod 131b constitute a rack and pinion structure. The second rod 131b moves along the longitudinal direction of the shaft 12 in conjunction with the rotation of the third gear 63.

The first and second rods 131a and 131b are disposed near the center of the shaft 12. More specifically, the first rod 131a and the second rod 131b are arranged such that the engagement position of the first rod 131a with the side surface of the disc of the second gear 62 as viewed from the first gear 61 is opposite to the engagement position of the second rod 131b with the side surface of the disc of the third gear 63 as viewed from the first gear 61. The second gear 62 engages with the first rod 131a on the right side of the second gear 62 as viewed from the first gear 61, whereas the third gear 63 engages with the second rod 131b on the left side of the third gear 63 as viewed from the first gear 61.

Thus, the first rod 131a and the second rod 131b move in the opposite directions along the longitudinal direction of the shaft 12, in conjunction with the rotation of the first gear 61.

The distal end portion 11 is manipulated by the first and second rods 131a and 131b which move in the opposite directions in conjunction with the rotation of the first gear 61. This configuration does not require a step of, e.g., wrapping the wire 14 around the actuation mechanism 15A and adjusting the wire tension. This configuration may further simplify the process of assembling the medical treatment tool 4b. Moreover, using the first and second rods 131a and 131b, which are rigid members, do not elongate unlike a flexible member, such as a wire which may elongate when used for a long time, and thus can secure the strength and durability.

The actuation mechanism 15A has, for example, three actuation units 101 each comprised of the first, second, and third gears 61, 62, and 63 described above. Hereinafter, the three actuation units 101 will be referred to as actuation units 101A, 101B, and 101C. The actuation units 101A, 101B, and 101C are disposed at substantially equal intervals in the circumferential direction of the shaft 12. In particular, the rotation axes of the first gears 61 of the respective actuation units 101 are arranged at substantially equal intervals.

The first gear 61, the second gear 62, and the third gear 63 included in the actuation unit 101A will be referred to as a first gear 61A, a second gear 62A, and a third gear 63A, respectively. The first gear 61, the second gear 62, and the third gear 63 included in the actuation unit 101B will be referred to as a first gear 61B, a second gear 62B, and a third gear 63B, respectively. The first gear 61, the second gear 62, and the third gear 63 included in the actuation unit 101C will be referred to as a first gear 61C, a second gear 62C, and a third gear 63C, respectively.

In the actuation unit 101A, the second gear 62A and the third gear 63A engage with the first gear 61A. The second gear 62A engages with the first rod 131a for the manipulation of the jaw 22. The third gear 63A engages with the second rod 131b for the manipulation of the jaw 22.

In the actuation unit 101B, the second gear 62B and the third gear 63B engage with the first gear 61B. The second gear 62B engages with the first rod 132a for the manipulation of the jaw 21. The third gear 63B engages with the second rod 132b for the manipulation of the jaw 21.

In the actuation unit 101C, the second gear 62C and the third gear 63C engage with the first gear 61C. The second gear 62C engages with the first rod 133a for the manipulation of the wrist portion 23. The third gear 63C engages with the second rod 133b for the manipulation of the wrist portion 23.

Referring again to FIG. 8, the actuation device 16 has four actuators 51, 52, 53, and 54 as the plurality of actuators 50. The actuators 51, 52, 53, and 54 may be servomotors, for example.

The actuation device 16 further includes a fourth gear 64, a fifth gear 65, a rotation mechanism 76, and a transmission member 78 that is a part of the transmitting portion 75A. The fourth gear 64 rotates the first gear 61C via the transmitting portion 75A. The fifth gear 65 engages with the fourth gear 64.

The actuator 51 rotates the first gear 61A via the transmitting portion 75A in accordance with a control signal transmitted from the patient-side apparatus 1 shown in FIG. 1. Similarly to the actuator 51, the actuator 52 rotates the first gear 61B via the transmitting portion 75A in accordance with a control signal transmitted from the patient-side apparatus 1 shown in FIG. 1.

The actuator 53 rotates the fifth gear 65 in accordance with a control signal transmitted from the patient-side apparatus 1. The fourth gear 64 rotates in conjunction with the rotation of the fifth gear 65, which further rotates the first gear 61C via the transmitting portion 75A.

The actuator 54 (which actuates the rotation mechanism) rotates a sixth gear 66, which serves as the rotation mechanism 76, in accordance with a control signal transmitted from the patient-side apparatus 1. The rotation of the sixth gear 66 causes the entire medical treatment tool 4b to rotate about an axis (i.e., a shaft axis) parallel to the longitudinal direction of the shaft 12.

Specifically, the rotation mechanism 76 further includes, in addition to the sixth gear 66, a disc-shaped rotation axis adjustment gear (a sun gear, not shown) that is fixed, with a side surface of the disc of the rotation axis adjustment gear engaging with the sixth gear 66. The central axis of the rotation axis adjustment gear coincides with the shaft axis. A rotational movement of the sixth gear 66 which revolves around the rotation axis adjustment gear (i.e., serves as a planetary gear) causes the distal end portion 11 of the medical treatment tool 4b to rotate about the shaft axis.

This configuration of the actuator 54 for rotating the shaft 12 in the directions arrows A enables appropriate changes of the orientation of the distal end portion 11.

The rotation mechanism 76 does not have to be a planetary gear mechanism as long as it is configured to be capable of rotating the medical treatment tool 4b about an axis parallel to the longitudinal direction (i.e., the shaft axis) of the shaft 12.

### [Variation of Actuation Mechanism]

The actuation mechanism 15A which actuates an end portion of the rigid elongate element has been described in FIG. 8. In a case of actuating an end portion of a flexible elongate element, an actuation mechanism 15B which will be described below may be used.

FIG. 10 is a diagram illustrating a perspective view of a configuration of a variation of the actuation mechanism of one or more embodiments.

Referring to FIG. 10, the actuation mechanism 15B has a housing 10, a plurality of actuation members 106 rotatably provided in the housing 10, a first gear 107 rotatably provided in the housing 10, a second gear 108 which engages with the first gear 107, an actuation transmitting system (not shown), and an actuator (not shown). In FIG. 10, an upper portion of the housing 10 is omitted to illustrate an internal configuration of the actuation mechanism 15B.

The actuation transmitting system has a plurality of transmission members which will be described later. Each of the plurality of actuation members 106 and the first gear 107 has a receiving member 122. Each of the plurality of transmission members engages with a corresponding one of these receiving members 122.

Each of the plurality of the actuation members 106 and the first gear 107 is rotatable about a rotation axis extending in a direction perpendicular to a surface of a base 36, that is, extending in the Y-axis direction. The second gear 108 is rotatable about a rotation axis extending in the longitudinal direction of the shaft 12, that is, extending in the Z-axis direction. The shaft 12 engages with the second gear 108, and rotates in the directions of arrows A in conjunction with the rotation of the second gear 108.

The actuator receives the control signal from the patient-side apparatus 1 illustrated in FIG. 1. Based on this control signal, the actuator rotates the plurality of transmission members. The rotation of each transmission member causes an associated one of the plurality of actuation members 106 and the first gear 107 to rotate.

More specifically, the actuation mechanism 15B has three actuation members 106. The three actuation members 106 will be referred to as actuation members 106A, 106B, and 106C. The actuation members 106A, 106B, and 106C are inserted, for example, in a plurality of through holes (not shown) formed in the base 36. In addition, a plurality of wires in the shaft 12, shown in FIG. 2, at a proximal end portion, opposite to the distal end portion 11, are wound around the actuation members 106A, 106B, and 106C, respectively.

When the rotation, by the actuator, of the transmission member associated with the actuation member 106A causes the actuation member 106A to rotate, the wire wound around the actuation member 106A moves along the Z-axis. When the rotation, by the actuator, of the transmission member associated with the actuation member 106B causes the actuation member 106B to rotate, the wire wound around the actuation member 106B moves along the Z-axis. When the rotation, by the actuator, of the transmission member associated with the actuation member 106C causes the actuation member 106C to rotate, the wire wound around the actuation member 106C moves along the Z-axis. The distal end portion 11 illustrated in FIGS. 2 to 4 and 7 is manipulated in conjunction with the movement of the elongate element 13 in this manner.

When the rotation, by the actuator, of the transmission member associated with the first gear 107 causes the first gear 107 to rotate, the second gear 108 engaged with the first gear 107 rotates about a rotation axis extending in the Z-axis direction. Consequently, the shaft 12 rotates in the directions of arrows A in conjunction with the rotation of the second gear 108.

### [Transmitting Portion]

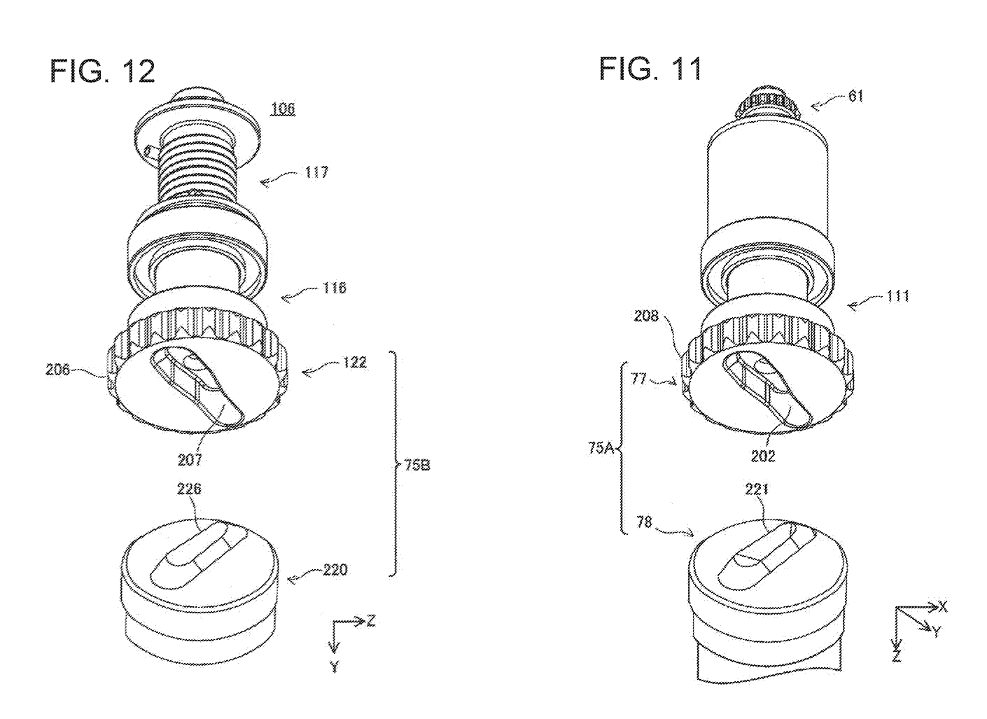

FIG. 11 is a diagram illustrating a perspective view of a configuration of a transmitting portion which transmits power between the actuation mechanism and the actuation device illustrated in FIG. 8.

Referring to FIG. 11, the transmitting portion 75A includes a receiving member 77 of the actuation mechanism 15A and the transmission member 78 of the actuation device 16.

The receiving member 77 is coupled to the first gear 61 via a rotation portion 111. The receiving member 77 engages with the transmission member 78, and rotates in conjunction with the rotation of the transmission member 78. The transmission member 78 is driven to rotate directly or indirectly by the actuator 50 shown in FIG. 8.

More specifically, the receiving member 77 has a rotatable disc portion 208. An engaged portion 202 is formed in a main surface of the disc portion 208. The transmission member 78 has an engaging portion 221 engageable with the engaged portion 202 of the receiving member 77.

The engaged portion 202 of the receiving member 77 is, for example, a groove or an indentation which, on the main surface of the disc portion 208, is asymmetric with respect to all straight lines passing through the center of said main surface, and has a continuous shape. The engaging portion 221 of the transmission member 78 is, for example, a projection which, on a main surface of the transmission member 220, is asymmetric with respect to all straight lines passing through the center of said main surface, and has a continuous shape, similarly to the engaged portion 202.

Thus, the orientation of the receiving member 77 for actuating the distal end portion 11 illustrated in FIG. 2 can be fixed at one specific position within 360 degrees.

The engaging portion 221 and the engaged portion 202 have a linear shape, for example. Further, for example, the engaging portion 221 is in a shape which passes through the center of the main surface of the transmission member 78, and the engaged portion 202 is in a shape which passes through the center of the main surface of the disc portion 208.

The engaging portion 221 is configured to be tapered such that the width thereof gradually decreases toward the receiving member 77 (i.e., toward the negative direction of the Z-axis shown in FIG. 11) in the Z-axis direction orthogonal to the main surface of the transmission member 78.

The transmission member 78 is biased toward the receiving member 77, that is, toward the negative direction of the Z-axis illustrated in FIG. 11, by a spring or the like. Thus, even if there is a small misalignment between the engaged portion 202 and the engaging portion 221, such a misalignment is corrected at the time of attachment, and the engaging portion 221 is fitted in the engaged portion 202. This configuration can simplify a step of engaging the engaging portion 221 with the engaged portion 202.

The engaging portion 221 may be configured as a groove or an indentation, instead of being configured as the projection. In a case in which the engaging portion 221 is a groove or an indentation, the engaged portion 202 is configured as a projection engageable with the engaging portion 221.

The shape of the engaged portion 202 is not limited to the shape described above, as long as the engaged portion 202 is asymmetric, on the plane of rotation of the disc portion 208, with respect to all straight lines passing through the center of the disc portion 208, and has a continuous shape.

The receiving member 77 and the transmission member 78 do not have to be engaged directly with each other to transmit the movement of the transmission member 78 to the receiving member 77. For example, the movement of the transmission member 78 may be transmitted to the receiving member 77 via an adapter with an integrally formed drape.

FIG. 12 is a diagram illustrating a perspective view of a configuration of a transmitting portion which transmits power between the actuation mechanism and the actuation device illustrated in FIG. 10.

Referring to FIG. 12, a rotation portion 116 of the actuation member 106 has the receiving member 122 that engages with the transmission member 220, as mentioned above. The transmission member 220 and the receiving member 122 constitute a transmitting portion 75B.

The receiving member 122 has a rotatable disc portion 206. An engaged portion 207 is formed in a main surface of the disc portion 206. The transmission member 220 has an engaging portion 226 engageable with the engaged portion 207 of the receiving member 122.

The configurations of the disc portion 206 and the engaged portion 207 of the receiving member 122 shown in FIG. 12 and the configuration of the engaging portion 226 of the transmission member 220 are similar to, or the same as, those of the disc portion 208 and the engaged portion 202 of the receiving member 77 shown in FIG. 11 and the engaging portion 221 of the transmission member 78. Thus, details will not be described.

### [Movement of Distal End Portion According to Rotation of First Gear]

Referring again to FIG. 8, the actuators 51, 52, and 53 respectively rotate the first gears 61A, 61B, and 61C of the actuation mechanism 15A in accordance with the control signal.

The rotation of the first gear 61A causes the second gear 62A and the third gear 63A to rotate. Accordingly, the first rod 131a engaged with the second gear 62A and the second rod 121b engaged with the third gear 63A move in the opposite directions from each other in the Z-axis direction. That is, the first rod 131a or the second rod 131b, when pulled in the Z1 direction, causes the jaw 22 to pivot in the C1a or C1b direction shown in FIG. 6.

Specifically, the first gear 61A, when rotated in the direction of arrow K11 shown in FIG. 8, causes the second gear 62A to rotate in the direction of arrow K12, which causes the first rod 131a engaged with the second gear 62A to move in the Z2 direction. In this case, the third gear 63A rotates in the direction of arrow K13, and the second rod 131b coupled to the third gear 63A moves in the Z1 direction.

The movement of the first rod 131a in the Z2 direction and the movement of the second rod 131b in the Z1 direction cause the jaw 22 to pivot in the C2b direction as shown in FIG. 6.

Likewise, the first gear 61A, when rotated in the direction of arrow K21, causes the second gear 62A to rotate in the direction of arrow K22, which causes the first rod 131a engaged with the second gear 62A to move in the Z1 direction. In this case, the third gear 63A rotates in the direction of arrow K23, and the second rod 131b coupled to the third gear 63A moves in the Z2 direction.

The movement of the first rod 131a in the Z1 direction and the movement of the second rod 131b in the Z2 direction cause the jaw 22 to pivot in the C2a direction as shown in FIG. 6.

In a case of rotation of the first gear 61B, too, similarly to the case of rotation of the first gear 61A, the second gear 62B and the third gear 63B rotate in conjunction with the rotation of the first gear 61B. Accordingly, the first rod 132a engaged with the second gear 62B and the second rod 132b engaged with the third gear 63B move in the opposite directions from each other in the Z-axis direction. Consequently, the jaw 21 pivots in the C2a or C2b direction, as illustrated in FIG. 6.

In a case of rotation of the first gear 61C, too, similarly to the case of rotations of the first gear 61A and the second gear 61B, the second gear 62C and the third gear 63C rotate in conjunction with the rotation of the first gear 61C. Accordingly, the first rod 133a engaged with the second gear 62C and the second rod 133b engaged with the third gear 63C move in the opposite directions from each other in the Z-axis direction. Consequently, the wrist portion 23 pivots in the B1 or B2 direction, as illustrated in FIG. 3.

Note that the actuation mechanism 15A is not limited to the configuration having three actuation units 101. For example, in the case of providing two sets of two rods moving in opposite directions in the Z-axis direction as shown in FIG. 7, the actuation mechanism 15A may have two actuation units 101 associated with the two sets of rods.

However, the actuation mechanism 15A having three actuation units 101 enables delicate surgical procedures, because the three members (i.e., the jaw 21 and 22 and the wrist portion 23) at the end portion 11 can be operated independently of one another. For this reason, in a preferred embodiment, the actuation mechanism 15A may have three actuation units 101.

Referring again to FIG. 10, in the actuation mechanism 15B, the transmission member 220 shown in FIG. 12 is rotated by an actuator (not shown), thereby controlling the rotation of the disc portion 206 and manipulating the distal end portion 11 of the medical treatment tool 4b. Specifically, the actuation members 106A, 106B, and 106C and the first gear 107 are rotated by four actuators via the respective transmitting portions 75B.

For example, the rotation of the actuation member 106A causes the jaw 22 shown in FIG. 6 to pivot. The rotation of the actuation member 106B causes the jaw 21 shown in FIG. 6 to pivot. The rotation of the actuation member 106C causes the wrist portion 23 shown in FIG. 6 to pivot. The rotation of the first gear 107 causes the shaft 12 shown in FIG. 10 to rotate.

Note that the actuation mechanism 15B is not limited to the configuration having three actuation members 106.

(Attachable/Detachable Mechanism)

The medical treatment tool 4b can be disassembled with respect to the shaft 12. That is, the medical treatment tool 4b is configured such that a unit adjacent to the distal end portion 11 and a unit adjacent to the actuation mechanism 15 are detachable from, and attachable to, the shaft 12.

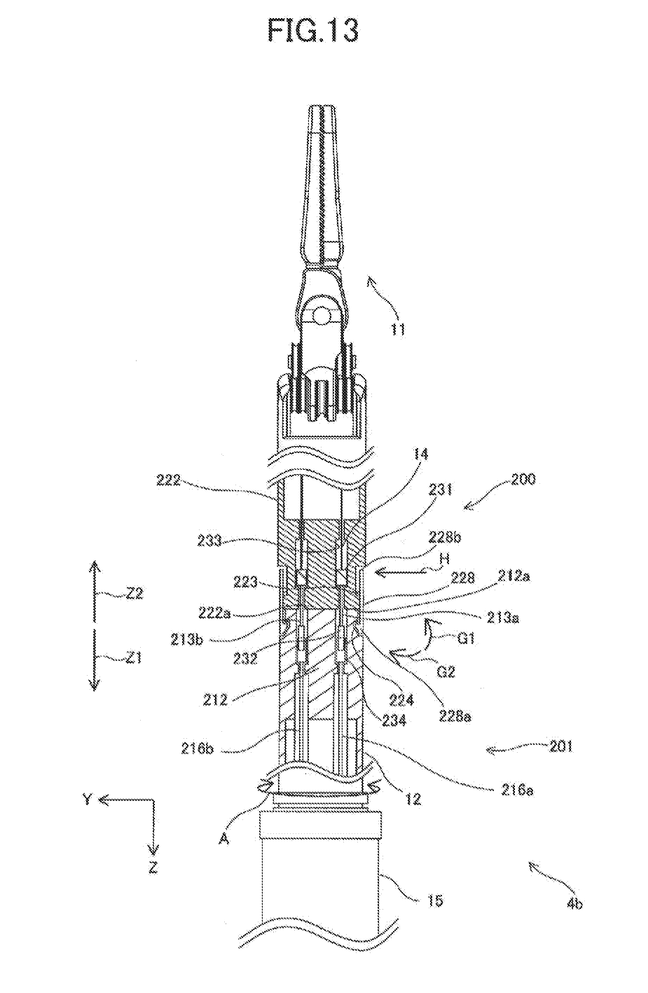

FIG. 13 is a diagram illustrating a cross-sectional view of a configuration (Example 1) of an attachable/detachable mechanism of the medical treatment tool.

Referring to FIG. 13, the unit adjacent to the distal end portion 11 includes a distal end-side shaft (a cylindrical portion) 222 attachable to an actuation-side shaft 212. The distal end-side shaft 222 and the actuation-side shaft 212 constitute the shaft 12 shown in FIG. 2.

A rigid elongate element or a combination of a rigid elongate element and a flexible elongate element is used, as will be described below, as an elongate element for use in the unit adjacent to the distal end portion 11 and the unit adjacent to the actuation mechanism 15 of the medical treatment tool 4b having an attachable/detachable mechanism. In particular, a combination of a rigid elongate element and a flexible elongate element can be used in the unit adjacent to the distal end portion 11.

In this embodiment, the elongate element shown in FIG. 5 is used as the elongate element 13 which manipulates the distal end portion 11. That is, the first rod 13a has a first distal end-side rod 213a and a first actuation-side rod 216a. The second rod 13b has a second distal end-side rod 213b and a second actuation-side rod 216b. The first distal end-side rod 213a and the second distal end-side rod 213b are connected to the wire 14.

The medical treatment tool 4b includes, for example, three sets of a pair of first distal end-side rod 213a and second distal end-side rod 213b, and three sets of a pair of first actuation-side rod 216a and second actuation-side rod 216b. FIG. 7 illustrates a pair of first distal end-side rod 213a and second distal end-side rod 213b, and a pair of first actuation-side rod 216a and second actuation-side rod 216b.

Hereinafter, a unit having the distal end portion 11, the distal end-side shaft 222, the first distal end-side rod 213a, the second distal end-side rod 213b, and the wire 14 will also be referred to as a “treatment tool unit 200.” A unit having the actuation-side shaft 212, the first actuation-side rod 216a, the second actuation-side rod 216b, and the actuation mechanism 15 will also be referred to as an “actuation unit 201.”

Each of the first distal end-side rod 213a and the second distal end-side rod 213b will also be referred to as a “distal end-side rod 213.” Each of the first actuation-side rod 216a and the second actuation-side rod 216b will also be referred to as an “actuation-side rod 216.”

The treatment tool unit 200 is configured to be attachable to the actuation unit 201. More specifically, the distal end-side shaft 222 of the treatment tool unit 200 is configured to be attachable to an end 212a of the actuation-side shaft 212 which is opposite to an end toward the actuation mechanism 15.

This configuration allows changes in the type of the surgical tool by simply detaching and attaching the treatment tool unit 200 from and to the actuation unit 201 without replacing the entire medical treatment tool 4b. It is therefore not necessary to prepare multiple types of the medical treatment tool 4b as a whole, which contributes to cost savings. Moreover, preparation of a spare of the treatment tool unit 200 for replacement in a surgery may reduce the necessity for the preparation of another medical treatment tool 4b as a spare, which contributes to keeping a space for a working space in the surgery.

That is, the distal end portion 11 can be attached to, and detached from, even a complex configuration which allows the distal end portion 11 to be manipulated in multiple degrees of freedom by the wire 14, which is a flexible elongate element connected to the distal end portion 11. This configuration enables complex manipulation of the distal end portion 11 without the necessity of replacing the entire medical treatment tool 4b, which contributes to achieving cost savings.

The treatment tool unit 200 further includes an engagement member 228.

An end (i.e., an opening) 222a of the distal end-side shaft 222 adjacent to the actuation mechanism 15 (hereinafter simply referred to as an end “toward the actuation mechanism 15”) in a state in which the treatment tool unit 200 is attached to the actuation-side shaft 212 has substantially the same diameter as the end 212a of the actuation-side shaft 212.

The engagement member 228 has substantially a rod shape and is connected to the distal end-side shaft 222 via a connecting portion 223. The engagement member 228 has an end toward the actuation mechanism 15, and the end is closer to the actuation mechanism 15 than the end 222a is. The engagement member 228 is pivotable on the connecting portion 223 in the direction of arrow G1 or G2.

The engagement member 228 has a first end (a claw) 228a toward the actuation mechanism 15. The first end 228a protrudes toward the central axis of the actuation-side shaft 212. The engagement member 228 is biased by, for example, a spring (not shown) so that the first end 228a pivots in the G2 direction, that is, toward the central axis of the actuation-side shaft 212.

The actuation-side shaft 212 accommodates three sets of the first actuation-side rod 216a and the second actuation-side rod 216b. The end 212a of the actuation-side shaft 212 is provided with a recess 224 engageable with the first end 228a of the engagement member 228 of the treatment tool unit 200.

(Attachment of Treatment Tool Unit to Actuation Unit)

In order to attach the treatment tool unit 200 to the actuation unit 201, a worker pushes a second end 228b of the engagement member 228 in a direction of arrow H. The pushing in the H direction causes the first end 228a of the engagement member 228 to pivot on the connecting portion 223 in the G1 direction.

In this state, in which the first end 228a is pivoted in the G1 direction, the end 222a of the distal end-side shaft 222 and the end 212a of the actuation-side shaft 212 are brought into contact with each other. Releasing of the second end 228b from the pressure toward the H direction causes the first end 228a to pivot on the connecting portion 223 in the G2 direction and engage with the recess 224. The treatment tool unit 200 is attached to the actuation unit 201 in this manner.

In order to release the treatment tool unit 200 from the state of being attached to actuation-side shaft 212, a worker pushes the second end 228b again in the H direction to pivot the first end 228a in the G1 direction. The first end 228a and the recess 224 are disengaged from each other by this pushing in the H direction, which allows the worker to move the treatment tool unit 200 in a direction away from the actuation unit 201, and release the treatment tool unit 200 from the state of attachment to the actuation unit 201.

(Coupling of Distal End-Side Rod and Actuation-Side Rod)

Each of the plurality of distal end-side rods 213 can be coupled to an associated one of the plurality of actuation-side rods 216.

More specifically, the distal end-side rod 213 has a thick portion (a first end) 231, which is an end toward the distal end portion 11, and a distal end-side coupling portion (a second end) 232, which is an end toward the actuation mechanism 15. In this embodiment, the distal end-side coupling portion 232 is a pin. Further, the actuation-side rod 216 has an actuation-side coupling portion 234, which is an end toward the distal end portion 11. In this embodiment, the actuation-side coupling portion 234 is a connector for receiving the pin 232.

The distal end-side shaft 222 is provided therein with a passage 233 as a distal end-side movement restricting portion. The passage 233 accommodates therein the thick portion 231 and restricts movement of the thick portion 231 in the Z-axis direction. The passage 233 allows the distal end-side rod 213 and the wire 14 to move therein. Each of both ends of the passage 233 has a diameter not large enough to allow passage of the thick portion 231.

Bringing the treatment tool unit 200 close to the actuation-side shaft 212 by the worker who is pushing the second end 228b of the engagement member 228 in the H direction to keep the first end 228a pivoted in the G1 direction, causes the pin 232 to come into contact with the connector 234, by which the pin 232 receives a force in the Z2 direction.

As a result, the distal end-side rod 213 moves in the Z2 direction until the thick portion 231 comes into contact with the end of the passage 233 toward the distal end portion 11, where the movement of the distal end-side rod 213 in the Z2 direction is restricted. When the worker brings the treatment tool unit 200 even closer to the actuation-side shaft 212 in this state, the pin 232 enters into the connector 234. The distal end-side rod 213 and the actuation-side rod 216 are fitted and coupled to each other in this manner.

When a worker releases the treatment tool unit 200 from the state of being attached to the actuation unit 201, the thick portion 231 and the pin 232 of the distal end-side rod 213, the engagement member 228, and the passage 233 in the distal end-side shaft 222 function as a decoupling mechanism for decoupling the distal end-side rod 213 and the actuation-side rod 216.

That is, bringing the treatment tool unit 200 in a direction away from the actuation-side shaft 212 by the worker who is pushing the first end 228a in the H direction, the pin 232 is pulled in the Z1 direction while the pin 232 is received in the connector 234.

As a result, the distal end-side rod 213 moves in the Z1 direction until the thick portion 231 of the distal end-side rod 213 comes into contact with the end of the passage 233 toward the actuation mechanism 151, where the movement of the distal end-side rod 213 in the Z1 direction is restricted. When the worker brings the treatment tool unit 200 further away from the actuation-side shaft 212 in this state, the pin 232 removes from the connector 234. The distal end-side rod 213 and the actuation-side rod 216 are decoupled from each other in this manner.

The pin 232 and the connector 234 (which are the end of the distal end-side rod 213 toward the actuation mechanism 15 and the end of the actuation-side rod 216 toward the distal end portion 11, respectively) enable easy coupling and decoupling between the distal end-side rod 213 and the actuation-side rod 216.

The end 222a of the distal end-side shaft 222 may have a diameter greater than the diameter of the other portion of the distal end-side shaft 222, that is, the portion except the end 222a. Such a configuration prevents the components, such as the pin 232 and the connector 234, for engaging the distal end-side rod 213 and the actuation-side rod 216 from having limited sizes, which facilitates the manufacture of the components and secures the coupling strength.

The mechanism for coupling the distal end-side rod 213 and the actuation-side rod 216 is not limited to the configuration using the pin 232 and the connector 234 as long as the mechanism has sufficient strength and durability.

For example, the end of the distal end-side rod 213 toward the actuation mechanism 15 may be formed of an elastic member and configured to be received in the end, configured as a connector, of the actuation-side rod 216 toward the distal end portion 11, while being deformed by a pushing force applied in a predetermined direction like an end portion of a local area network (LAN) cable. In this case, when the distal end-side rod 213 reaches a predetermined position with respect to the actuation-side rod 216, the end of the distal end-side rod 213 received in the connector returns to its original shape in the connector, and engages with, for example, a groove formed in the connector.

The treatment tool unit 200 may be capable of being divided into a plurality of members. For example, the distal end-side shaft 222 may be divided into a member toward the distal end portion 11 and a member toward the actuation mechanism 15. The distal end-side rod 213 received in the distal end-side shaft 222 may also be divided into a member toward the distal end portion 11 and a member toward the actuation mechanism 15. Thus, the treatment tool unit 200 may be divided into two members 27.

Now, another embodiment will be described with reference to the drawings. Note that the same or corresponding parts are denoted by the same reference characters in the drawings, and the description thereof will not be repeated.

### Second Embodiment

In the above-mentioned medical treatment tool 4b according to the first embodiment, the pin 232 of the distal end-side rod 213 is received in the connector 234 of the actuation-side rod 216, thereby coupling the distal end-side rod 213 and the actuation-side rod 216 to each other.

In contrast, in the medical treatment tool 4b according to the second embodiment, both of a distal end-side coupling portion 251 at the end of the distal end-side rod 213 and an actuation-side coupling portion 252 at the end of the actuation-side rod 216 have a protruded shape. The distal end-side rod 213 and the actuation-side rod 216 are coupled to each other by engagement of the distal end-side coupling portion 251 and the actuation-side coupling portion 252 with each other.

### [Configuration of Medical Treatment Tool]

(Attachment of Treatment Tool Unit to Shaft)

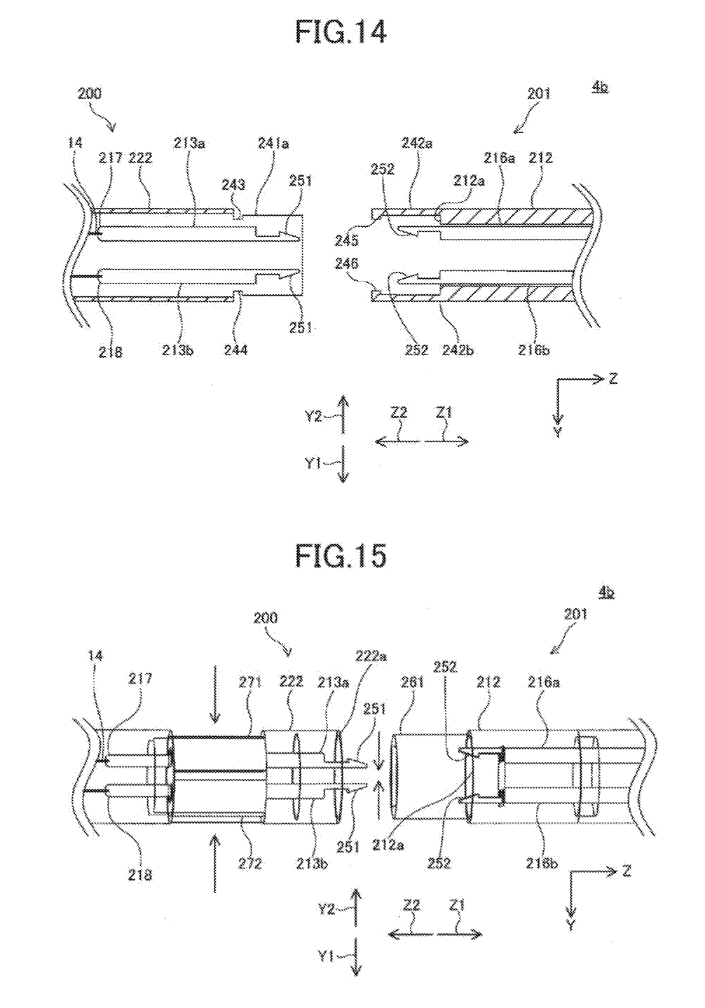

FIG. 14 is a diagram illustrating a cross-sectional view of a configuration (Example 2) of an attachable/detachable mechanism of the medical treatment tool. In FIG. 14, the arrow Y1 indicates the positive direction of the Y-axis, and the arrow Y2 indicates the negative direction of the Y-axis. FIG. 14 illustrates a pair of first distal end-side rod 213a and second distal end-side rod 213b, and a pair of first actuation-side rod 216a and second actuation-side rod 216b.

Referring to FIG. 14, the distal end-side shaft 222 of the treatment tool unit 200 has two first extending portions 241a and 241b extending toward the actuation mechanism 15. The two first extending portions 241a and 241b are arranged in a direction perpendicular to a YZ plane. FIG. 14 illustrates the first extending portion 241a, and the first extending portion 241b is not shown.

The actuation-side shaft 212 has two second extending portions 242a and 242b extending from the end 212a toward the distal end portion 11. The two second extending portions 242a and 242b are arranged in a direction parallel to the YZ plane.

The first extending portions 241a and 241b of the treatment tool unit 200 and the second extending portions 242a and 242b of the actuation-side shaft 212 are connected to one another, thereby forming an outer periphery of the actuation-side shaft 212.

Each of the first extending portions 241a and 241b has a recess 243 at a portion to be coupled to the second extending portion 242a, and a recess 244 at a portion to be coupled to the second extending portion 242b. The second extending portion 242a has a projection 245 which can be engaged with the recess 243. The second extending portion 242b has a projection 246 which can be engaged with the recess 244.

In order to attach the treatment tool unit 200 to the actuation unit 201, a worker moves the treatment tool unit 200 in the direction perpendicular to the YZ plane so that the projection 245 engages with the recess 243, and that the projection 246 engages with the recess 244. The treatment tool unit 200 is attached to the actuation unit 201 in this manner.

In order to release the treatment tool unit 200 from the state of being attached to the actuation unit 201, a worker moves the treatment tool unit 200 in the direction perpendicular to the YZ plane. The projections 245 and 246 and the recesses 243 and 244 are disengaged from each other by this movement in said direction, which allows the worker to release the treatment tool unit 200 from the state of attachment to the actuation unit 201.

(Coupling of Distal End-Side Rod and Actuation-Side Rod)

The treatment tool unit 200 has three sets of the first distal end-side rod 213a, the second distal end-side rod 213b, and the wire 14. The first distal end-side rods 213a included in the three sets are arranged next to one another so as to be perpendicular to the YZ plane. The second distal end-side rods 213b included in the three sets are also arranged next to one another so as to be perpendicular to the YZ plane.

The first distal end-side rod 213a and the second distal end-side rod 213b are connected to the wire 14. The end 217 of the first distal end-side rod 213a toward the wire 14 and the end 218 of the second distal end-side rod 213b toward the wire 14 are tapered.

Such a configuration prevents the wire 14 from being damaged by an angular edge of a different distal end-side rod 213 coupled to another wire 14, even when the wire 14 is driven and comes into contact with the edge of the different distal end-side rod 213.

Each of the first distal end-side rod 213a and the second distal end-side rod 213b has a distal end-side coupling portion 251 at the end toward the actuation mechanism 15. Each distal end-side coupling portion 251 has a protrusion which protrudes along the same straight line. Specifically, the distal end-side coupling portion 251 of the first distal end-side rod 213a has a protrusion which protrudes, e.g., in the Y2 direction, and is formed into a tapered hook shape. The distal end-side coupling portion 251 of the second distal end-side rod 213b has a protrusion which protrudes, e.g., in the Y1 direction, and is formed into a tapered hook shape.

The medical treatment tool 4b has three sets of a pair of the first actuation-side rod 216a and the second actuation-side rod 216b. The first actuation-side rods 216a included in the three sets are arranged next to one another so as to be perpendicular to the YZ plane. The second actuation-side rods 216b included in the three sets are also arranged next to one another so as to be perpendicular to the YZ plane.

Each of the first and second actuation-side rods 216a and 216b has an actuation-side coupling portion 252 at an end adjacent to the distal end portion 11 (hereinafter simply referred to as an end “toward the distal end portion 11”) in a state in which the treatment tool unit 200 is attached to the actuation-side shaft 212. Each actuation-side coupling portion 252 has a protrusion which protrudes along the same straight line, and is formed into a tapered hook shape. Specifically, the actuation-side coupling portion 252 of the first actuation-side rod 216a has a protrusion which protrudes, e.g., in the Y1 direction. The actuation-side coupling portion 252 of the second actuation-side rod 216b has a protrusion which protrudes, e.g., in the Y2 direction, and is formed into a tapered hook shape.

The actuation-side coupling portion 252 and an associated one of the distal end-side coupling portions 251 are fitted to each other when a worker moves the treatment tool unit 200 in the direction perpendicular to the axial direction of the distal end-side rod 213, that is, in the direction perpendicular to the YZ direction, in order to attach the treatment tool unit 200 to the actuation unit 201, as described above. The distal end-side rod 213 and the actuation-side rod 216 are coupled to each other in this manner.

When a worker releases the treatment tool unit 200 from the state of being attached to the actuation unit 201, the distal end-side coupling portion 251 and the actuation-side coupling portion 252 function as a decoupling mechanism for decoupling the distal end-side rod 213 and the actuation-side rod 216.

That is, when a worker moves the treatment tool unit 200 in the direction perpendicular to the YZ plane to release the treatment tool unit 200 from the state of being attached to the actuation unit 201, the distal end-side coupling portion 251 and the actuation-side coupling portion 252 are disengaged from each other, making it possible to decouple the distal end-side rod 213 and the actuation-side rod 216 from each other.

The other configurations and operations are the same as those described in the first embodiment. Detailed description thereof will not be repeated here.

### Third Embodiment

A medical treatment tool 4b according to a third embodiment is configured such that a portion of the outer periphery of the distal end-side shaft 222 is pushed to move the distal end-side rod 213 for engagement with the actuation-side rod 216.

### [Configuration of Medical Treatment Tool]

(Attachment of Treatment Tool Unit to Actuation Unit)

FIG. 15 is a diagram illustrating a perspective view of a configuration (Example 3) of an attachable/detachable mechanism of the medical treatment tool. FIG. 15 illustrates a pair of first distal end-side rod 213a and second distal end-side rod 213b, and a pair of first actuation-side rod 216a and second actuation-side rod 216b.

Referring to FIG. 15, the actuation-side shaft 212 has a fitting portion 261 extending toward the distal end portion 11 from the end 212a. The fitting portion 261 has a cylindrical shape. The outer diameter of the fitting portion 261 is slightly smaller than the inner diameter of the end 222a of the distal end-side shaft 222 of the treatment tool unit 200. Thus, bringing the treatment tool unit 200 close to the actuation unit 201 to make the fitting portion 261 fitted in the end 222a, for example, allows the treatment tool unit 200 to be attached to the actuation unit 201.

In order to release the treatment tool unit 200 from the state of being attached to the actuation unit 201, a worker moves the treatment tool unit 200 in a direction away from the actuation unit 201, which allows the worker to release the treatment tool unit 200 from the state of attachment to the actuation unit 201.

(Coupling of Distal End-Side Rod and Actuation-Side Rod)

Similarly to the treatment tool unit 200 according to the second embodiment illustrated in FIG. 14, the treatment tool unit 200 has three sets of the first distal end-side rod 213a, the second distal end-side rod 213b, and the wire 14. The first distal end-side rods 213a included in the three sets are arranged next to one another so as to be perpendicular to the YZ plane. The second distal end-side rods 213b included in the three sets are also arranged next to one another so as to be perpendicular to the YZ plane.

The first distal end-side rod 213a and the second distal end-side rod 213b are connected to the wire 14. The end 217 of the first distal end-side rod 213a toward the wire 14 and the end 218 of the second distal end-side rod 213b toward the wire 14 are tapered.

Each of the first distal end-side rod 213a and the second distal end-side rod 213b has a distal end-side coupling portion 251 at the end toward the actuation mechanism 15. The distal end-side coupling portion 251 of the first distal end-side rod 213a has a protrusion which protrudes, e.g., in the Y2 direction, and is formed into a tapered hook shape. The distal end-side coupling portion 251 of the second distal end-side rod 213b has a protrusion which protrudes, e.g., in the Y1 direction, and is formed into a tapered hook shape.

The three first distal end-side rods 213a are fixable to a force-applying portion 271 which forms a portion of the outer periphery of the distal end-side shaft 222. The force-applying portion 271 is movable toward the inside of the distal end-side shaft 222, and is biased toward the outside, i.e., in the Y2 direction, by a spring (not shown), for example.

The three second distal end-side rods 213b are fixable to a force-applying portion 272 which forms a portion of the outer periphery of the distal end-side shaft 222. The force-applying portion 272 is movable toward the inside of the distal end-side shaft 222, and is biased toward the outside, i.e., in the Y1 direction, by a spring (not shown), for example.

The medical treatment tool 4b has three sets of a pair of the first actuation-side rod 216a and the second actuation-side rod 216b. The first actuation-side rods 216a included in the three sets are arranged next to one another so as to be perpendicular to the YZ plane. The second actuation-side rods 216b included in the three sets are also arranged next to one another so as to be perpendicular to the YZ plane.

Each of the first and second actuation-side rods 216a and 216b has an actuation-side coupling portion 252 at an end toward the distal end portion 11. The actuation-side coupling portion 252 of the first actuation-side rod 216a has a protrusion which protrudes, e.g., in the Y1 direction, and is formed into a tapered hook shape. The actuation-side coupling portion 252 of the second actuation-side rod 216b has a protrusion which protrudes, e.g., in the Y2 direction, and is formed into a tapered hook shape.

In order to attach the treatment tool unit 200 to the actuation unit 201, a worker fits the treatment tool unit 200 onto the fitting portion 261, while pushing the force-applying portions 271 and 272 in the Y1 and Y2 directions, respectively. Pushing of the force-applying portions 271 and 272 causes a distal end-side movement restricting portion to function. The functioning of the distal end-side movement restricting portion restricts the movements of the first and second distal end-side rods 213a and 213b along the Z-axis direction. It is therefore possible to fit distal end-side shaft 222 onto the fitting portion 261, while maintaining the position of the distal end-side coupling portion 251.

Examples of the distal end-side movement restricting portion which functions through the pushing of the force-applying portions 271 and 272 may include projections (not shown) which engage with notches (not shown) formed at the first and second distal end-side rods 213a and 213b, and an elastic member (not shown), such as a rubber, which obstructs movements of the first and second distal end-side rods 213a and 213b due to the friction caused by the pushing of the force-applying portions 271 and 272.

The distal end-side coupling portion 251 of the first distal end-side rod 213a and the distal end-side coupling portion 251 of the second distal end-side rod 213b are tapered. Thus, the two distal end-side coupling portions 251, even when they are brought into contact with, for example, the actuation-side coupling portions 252 during the movement of the distal end-side shaft 222 in the Z1 direction, appropriately move toward each other in the Y-axis directions, and are engaged and coupled with the actuation-side coupling portions 252 in the end.

In order to attach the treatment tool unit 200 to the actuation unit 201 more reliably, a worker may push the force-applying portion 271 in the Y1 direction and the force-applying portion 272 in the Y2 direction as illustrated in FIG. 15. Accordingly, the distal end-side coupling portion 251 of the first distal end-side rod 213a moves in the Y1 direction, and the distal end-side coupling portion 251 of the second distal end-side rod 213b moves in the Y2 direction.

In this state, the worker moves the treatment tool unit 200 close to the actuation-side shaft 212 up to a position where the distal end-side coupling portions 251 and the actuation-side coupling portions 252 overlap in the Y-axis direction, at which position the worker stops pushing the force-applying portions 271 and 272, and the distal end-side coupling portions 251 engage with the corresponding actuation-side coupling portions 252. The distal end-side rod 213 and the actuation-side rod 216 are engaged with each other in this manner.

The actuation-side rod 216 is movable in the Z1 direction and the Z2 direction by being actuated by the actuation mechanism 15A.

When a worker releases the treatment tool unit 200 from the state of being attached to the actuation unit 201, the distal end-side coupling portion 251, the actuation-side coupling portion 252, and the force-applying portions 271 and 272 function as a decoupling mechanism for decoupling the distal end-side rod 213 and the actuation-side rod 216.

That is, pushing, by a worker, of the force-applying portion 271 in the Y1 direction and the force-applying portion 272 in the Y2 direction so as to release the treatment tool unit 200 from the state of being attached to the actuation unit 201 disengages the distal end-side coupling portion 251 and the actuation-side coupling portion 252 from each other. When the worker brings the treatment tool unit 200 away from the actuation unit 201 in this state, the distal end-side rod 213 and the actuation-side rod 216 are decoupled from each other.

Note that the attachable/detachable mechanism described in the present embodiment is also applicable to a case where the treatment tool unit 200 does not include the wire 14. In this case, the distal end portion 11 is connected directly to the first and second distal end-side rods 213a and 213b, and is operated in conjunction with the actuation of the first and second distal end-side rods 213a and 213b.

### [Variation]

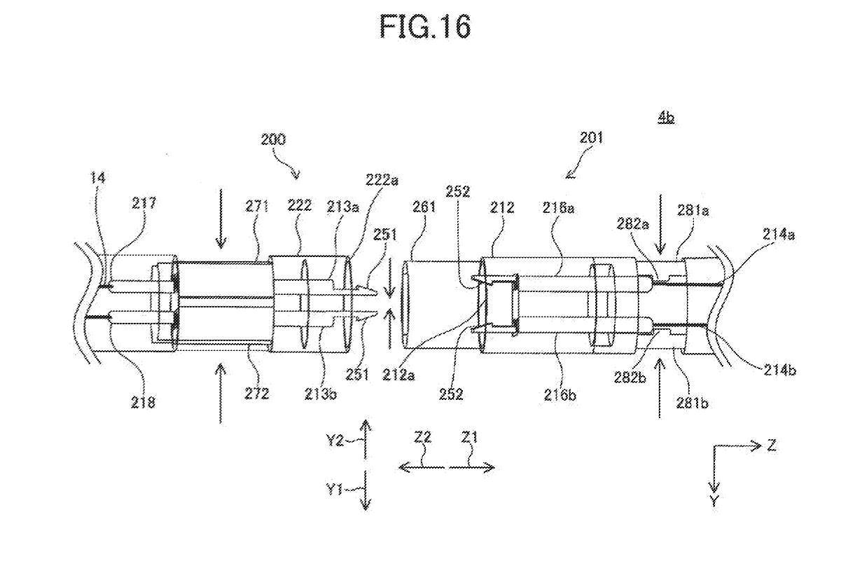

FIG. 16 is a diagram illustrating a perspective view of a variation of the configuration (Example 3) of the attachable/detachable mechanism of the medical treatment tool.

Referring to FIG. 16, an end of the first actuation-side rod 216a toward the actuation mechanism 15 may be connected to a wire 214a which is a flexible elongate element. An end of the second actuation-side rod 216b toward the actuation mechanism 15 may be connected to a wire 214b which is a flexible elongate element. The wires 214a and 214b are housed in the actuation-side shaft 212, and are wound around the actuation member 106 of the actuation mechanism 15B illustrated in FIGS. 10 and 12, for example.

In this configuration (in which the component coupled to the distal end-side rod 213 is the rigid actuation-side rod 216 and in which the wire 214a or the wire 214b is connected to the actuation-side rod 216 in the actuation mechanism 15B), such a component as a gear is not required unlike the case in which the actuation-side rod 216 is driven directly in the actuation mechanism 15, which allows a reduction in size of the medical treatment tool 4b.

The actuation-side shaft 212 has push portions 281a and 281b movable toward the inside of the actuation-side shaft 212. The push portion 281a is biased toward the outside, i.e., in the Y2 direction, by a spring (not shown), for example. The push portion 281a has a protrusion 282a protruding in the Y1 direction.

The push portion 281b is biased toward the outside, i.e., in the Y1 direction, by a spring (not shown), for example. The push portion 281b has a protrusion 282b protruding in the Y2 direction.

In order to engage the distal end-side rod 213 and the actuation-side rod 216 with each other, a worker pushes the push portion 281a in the Y1 direction and the push portion 281b in the Y2 direction. In this state, the worker brings the treatment tool unit 200 close to the actuation unit 201 so as to couple the distal end-side coupling portion 251 of the distal end-side rod 213 and the actuation-side coupling portion 252 of the actuation-side rod 216 to each other.

At this moment, the first actuation-side rod 216a receives a force in the Z1 direction due to the first distal end-side rod 213a brought into contact with the first actuation-side rod 216a. However, the movement of the first actuation-side rod 216a in the Z1 direction is restricted by the protrusion 282a of the push portion 281a which is in contact with the first actuation-side rod 216a. The second actuation-side rod 216b receives a force in the Z1 direction due to the second distal end-side rod 213b brought into contact with the second actuation-side rod 216b. However, the movement of the second actuation-side rod 216b in the Z1 direction is restricted by the protrusion 282b of the push portion 281b.

In this manner, the distal end-side rod 213 is coupled to the actuation-side rod 216 while the movement of the actuation-side rod 216 in the Z1 direction is restricted, which allows the distal end-side rod 213 and the actuation-side rod 216 to be easily coupled to each other even in a condition in which the end of the actuation-side rod 216 toward the actuation mechanism 15 is connected to the flexible wire 214.

When the worker eases the pushing force to the push portions 281a and 281b in the state in which the distal end-side rod 213 and the actuation-side rod 216 are coupled to each other, the push portion 281a moves in the Y2 direction and the push portion 281b in the Y1 direction. The protrusion 282a moves to a position away from the actuation-side rod 216a, and the protrusion 282b moves to a position away from the actuation-side rod 216b.

The actuation-side rods 216a and 216b are now movable in the Z1 direction and the Z2 direction by being actuated by the actuation mechanism 15B.

The other configurations and operations are the same as those described in the first and second embodiments. Detailed description thereof will not be repeated here.

The distal end portion 11 may preferably be in a standard posture when the treatment tool units 200 of the second and third embodiments and the variation of the third embodiment are attached and detached, as will be described in a fourth embodiment. The distal end portion 11 taking the standard posture allows the distal end-side coupling portion 251 and the actuation-side coupling portion 252 to be at standard positions, which facilitates the attaching and detaching of the treatment tool unit 200.

### Fourth Embodiment

In a medical treatment tool 4b according to a fourth embodiment, the distal end-side rod 213 and the actuation-side rod 216 are coupled to each other while the movement of the distal end-side rod 213 is restricted by a distal end-side movement restricting portion 430 provided in the distal end-side shaft 222 and the movement of the actuation-side rod 216 is restricted by an actuation-side movement restricting portion 424 provided in the actuation-side shaft 212.

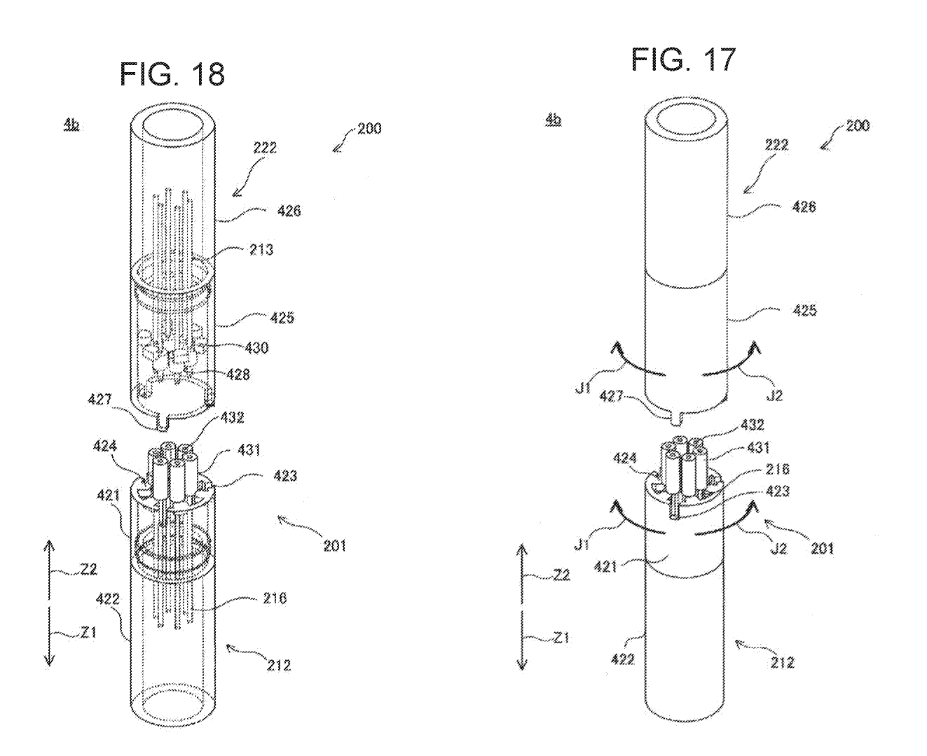

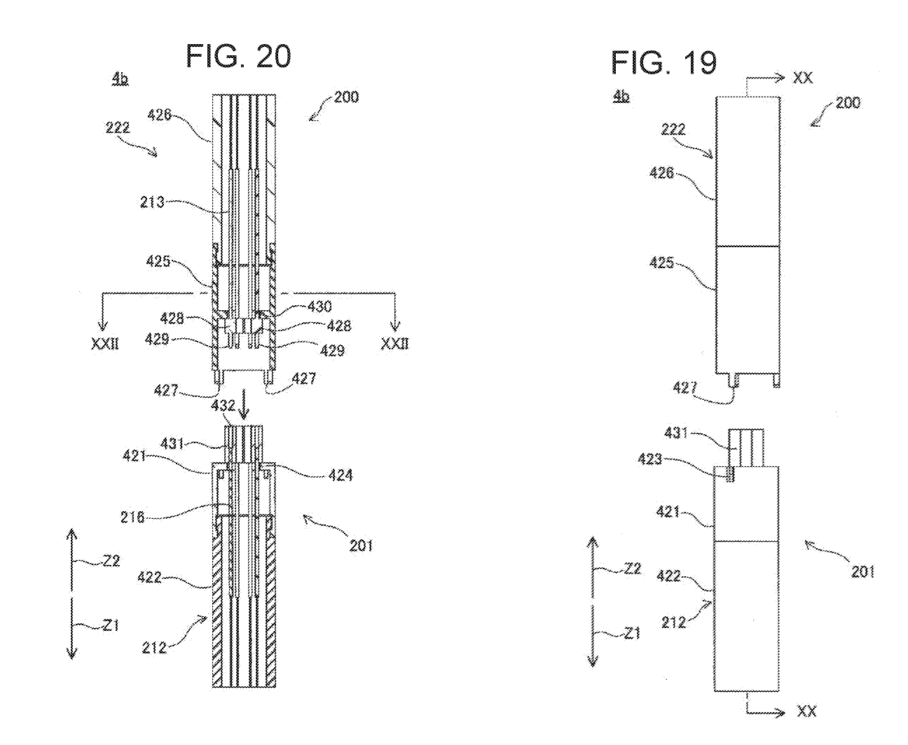

FIG. 17 is a diagram illustrating a perspective view of an external configuration (Example 4) of an attachable/detachable mechanism of the medical treatment tool. FIG. 18 is a diagram illustrating a perspective view of an internal configuration of the medical treatment tool in FIG. 17. FIG. 19 is a diagram illustrating a front view of the medical treatment tool in FIG. 17. FIG. 20 is a diagram illustrating a cross-sectional view taken along the line XX-XX of FIG. 19.

FIGS. 17 to 20 illustrate the distal end-side shaft 222, the actuation-side shaft 212, the distal end-side rod 213, and the actuation-side rod 216 of the medical treatment tool 4b.

Referring to FIGS. 17 and 18, the actuation-side shaft 212 includes a main body 422 and an outer shell 421 which covers a portion of the main body 422 at the distal end. The outer shell 421 is configured to be rotatable with respect to the main body 422 in directions of arrows J1 and J2 in FIG. 17. The rotatable outer shell 421 constitutes an actuation-side rotatable portion.

Note that the actuation-side rotatable portion does not necessarily have to cover a portion of the main body 422 as long as the outer shell 421 is rotatable with respect to the main body 422. The actuation-side rotatable portion may be an outer shell 421 rotatably arranged adjacent to the main body 422.

In addition, the outer shell 421 is provided with a recess 423 at a side where the treatment tool unit 200 is attached, that is, at the end toward the distal end. The recess 423 is recessed toward the side opposite from the distal end. The recess 423 includes, for example, three recesses formed at equal intervals. The outer shell 421 is also provided with an actuation-side movement restricting portion (an actuation-side projection) 424 at the end toward the distal end. The projection 424 protrudes toward the inside of the actuation-side shaft 212. The actuation-side movement restricting portion 424 includes, for example, six actuation-side movement restricting portions formed at equal intervals in the circumferential direction of the outer shell 421.

Each of the actuation-side rods 216 has an actuation-side coupling portion 431 at the end of the side where the distal end-side shaft 222 is attached. The actuation-side coupling portion 431 is thicker than the other portion of the actuation-side rod. The movement of the actuation-side coupling portion 431 of each of the six actuation-side rods 216 in the Z1 direction, that is, in the direction toward the actuation side along the shaft axis is restricted when the actuation-side coupling portion 431 abuts on a corresponding one of the six actuation-side movement restricting portions 424. Each actuation-side coupling portion 431 is provided with a hole 432 at a side toward the distal end.

A wire actuated by the actuation mechanism 15 (not shown) is connected to an end of each actuation-side rod 216 opposite to the end where the actuation-side coupling portion 431 is provided.

The distal end-side shaft 222 includes a main body 426 and an outer shell 425 which covers a portion of the main body 426 at an end opposite to the distal end. The outer shell 425 is configured to be rotatable with respect to the main body 426 in the directions of arrows J1 and J2 in FIG. 17. The rotatable outer shell 425 constitutes a distal end-side rotatable portion.

Note that the distal end-side rotatable portion does not necessarily have to cover a portion of the main body 426 as long as the outer shell 425 is rotatable with respect to the main body 426. The distal end-side rotatable portion may be an outer shell 425 rotatably arranged adjacent to the main body 426.

In addition, the outer shell 425 is provided with a protrusion 427 which protrudes toward the side where the actuation unit 201 is attached. The protrusion 427 includes, for example, three protrusions formed at equal intervals, which respectively engage with the three recesses 423 of the outer shell 421. The outer shell 425 is also provided with a distal end-side movement restricting portion (a distal end-side projection) 430 which protrudes toward the inside of the distal end-side shaft 222, as illustrated in FIG. 20. The distal end-side movement restricting portion 430 includes, for example, six distal end-side movement restricting portions formed at equal intervals.

In a preferred embodiment, the number of the distal end-side movement restricting portions 430 and the actuation-side movement restricting portions 424 is the same as the number of elongate elements passing through the portion where the treatment tool unit 200 and the actuation unit 201 are attached to and detached from each other.

The distal end-side rod 213 has a distal end-side coupling portion 428 at the end of the side where the actuation unit 201 is attached. The distal end-side coupling portion 428 is thicker than the other portion of the distal end-side rod. The movement of the distal end-side coupling portion 428 of each of the six distal end-side rods 213 in the Z2 direction, that is, in the direction toward the distal end along the shaft axis is restricted when the distal end-side coupling portion 428 abuts on a corresponding one of the six distal end-side movement restricting portions 430.

Each distal end-side coupling portion 428 is provided with a projection 429 which extends in the axial direction of the distal end-side shaft 222. The projection 429 is inserted in the hole 432 of the actuation-side coupling portion 431. The distal end-side coupling portion 428 and the actuation-side coupling portion 431 are coupled to each other in this manner.

An elongate element, such as a wire, which manipulates the distal end portion 11 is connected to an end of each distal end-side rod 213 opposite to the end where the distal end-side coupling portion 428 is provided.

In order to attach the treatment tool unit 200 to the actuation unit 201, a worker fits the three protrusions 427 of the distal end-side shaft 222 into the three recesses 423 of the actuation unit 201, allowing the outer shell 425 and the outer shell 421 to rotate in conjunction with each other.

The worker also fits the six projections 429 in the six holes 432, allowing the six distal end-side rods 213 to be coupled to the six actuation-side rods 216.

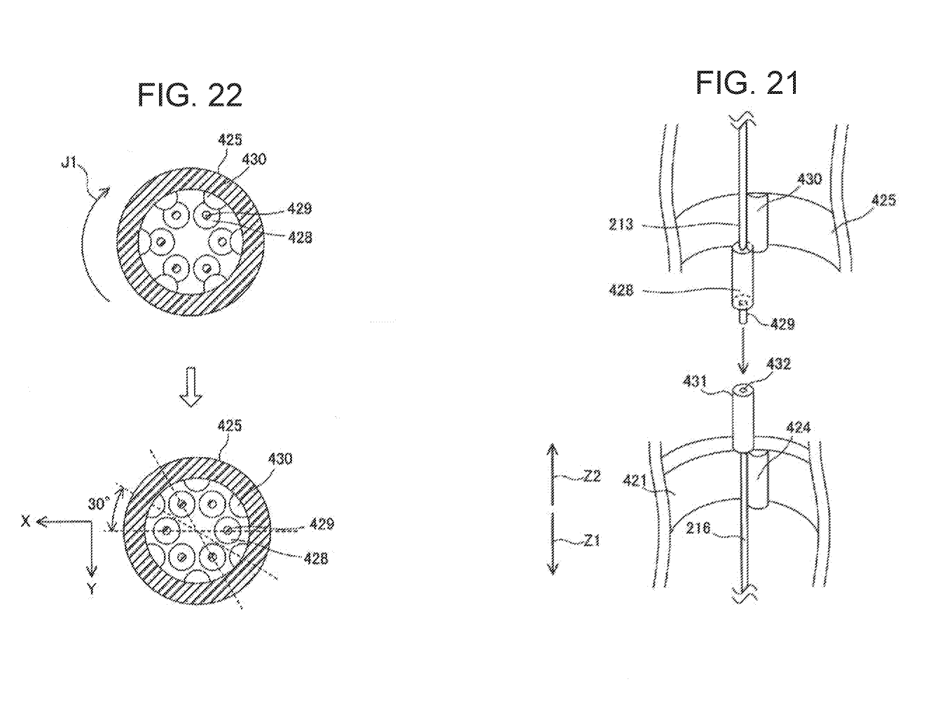

FIG. 21 is a diagram illustrating a perspective view of a state in which a distal end-side rod and an actuation-side rod, which are shown in FIG. 17, are about to be coupled to each other.

Referring to FIG. 21, in order to couple the distal end-side rod 213 and the actuation-side rod 216 to each other, a worker inserts the projection 429 of each of the six distal end-side rods 213 in the hole 432 of each of the six actuation-side rods 216. At this moment, as described above, the movement of the distal end-side rod 213 in the Z2 direction is restricted by the distal end-side coupling portion 428 abutting on the distal end-side movement restricting portion 430, and the movement of the actuation-side rod 216 in the Z1 direction is restricted by the actuation-side coupling portion 431 abutting on the actuation-side movement restricting portion 424.

The movement of the distal end-side rod 213 in the Z2 direction is restricted, and the movement of the actuation-side rod 216 in the Z1 direction is restricted in this manner. This configuration allows the distal end-side rod 213 and the actuation-side rod 216 to be coupled to each other even in the case in which the end of the distal end-side rod 213 toward the distal end portion 11 and the end of the actuation-side rod 216 toward the actuation mechanism 15 are connected to flexible elongate elements, such as wires.

The worker rotates the outer shells 425 and 421 in the J1 direction shown in FIG. 17 while the distal end-side shaft 222 and the actuation-side shaft 212 are fitted to each other and the distal end-side rod 213 and the actuation-side rod 216 are coupled to each other. The distal end-side movement restricting portion 430 and the actuation-side movement restricting portion 424 rotate accordingly in conjunction with each other, and move to positions away from the distal end-side coupling portion 428 and the actuation-side coupling portion 431, respectively.

FIG. 22 is a diagram illustrating cross-sectional views taken along the line XXII-XXII of FIG. 20.

Referring to FIG. 22, in a state in which the distal end-side rod 213 and the actuation-side rod 216 are coupled to each other, each distal end-side coupling portion 428 is in contact with the distal end-side movement restricting portion 430 as illustrated in the upper cross-sectional view of FIG. 22, and the movement of each of the six distal end-side rods 213 in the Z2 direction is restricted. Rotation in this state, by a worker, of the outer shell 425 and/or the outer shell 421 in the J1 direction by a predetermined angle causes the six distal end-side movement restricting portions 430 to rotate in the J1 direction by the predetermined angle, without changes in the positions of the six distal end-side coupling portions 428, and leads the distal end-side movement restricting portions 430 to positions where the distal end-side movement restricting portions 430 do not overlap with the distal end-side coupling portions 428 with respect to the XY plane, as illustrated in the lower cross-sectional view of FIG. 22.

Note that the treatment tool unit 200 is provided with a stopper (not shown) to prevent the outer shell 425 from being rotated by more than the predetermined angle when the outer shell 425 is rotated in conjunction with the outer shell 421.

The necessary predetermined angle for rotation differs depending on the number of elongate elements provided inside the outer shell 425. In the present embodiment, there are six elongate elements provided inside the outer shell 425, and these elongate elements are arranged at equal intervals along a circular pattern. Thus, the predetermined angle is set to be 30° (360°×⅙×½). The stopper is arranged so that the outer shell 425 is not rotated by more than 30°.

In a case in which four elongate elements are provided inside the outer shell 425, the predetermined angle may be set to be 45° (360°×¼×½). That is, the angle to be determined as the predetermined angle can be expressed by 360°×1/n×½, where n is the number of elongate elements provided inside the outer shell 425. Further, the stopper is arranged so that the outer shell 425 is not rotated by more than the predetermined angle in both of the J1 and J2 directions.

Regarding the actuation-side rod 216, as well, which is illustrated in FIG. 17, in a state in which the distal end-side rod 213 and the actuation-side rod 216 are coupled to each other, the actuation-side coupling portion 431 of each of the six actuation-side rods 216 is in contact with a corresponding one of the six actuation-side movement restricting portions 424, and the movement of the actuation-side coupling portion 431 in the Z1 direction is restricted. Rotation in this state, by a worker, of the outer shell 425 and/or the outer shell 421 in the J1 direction by a predetermined angle causes the six actuation-side movement restricting portions 424 to rotate in the J1 direction by the predetermined angle, without changes in the positions of the six actuation-side coupling portions 431, and leads the actuation-side movement restricting portions 424 to positions where the actuation-side movement restricting portions 424 do not overlap with actuation-side coupling portions 431 with respect to the XY plane.

The distal end-side rod 213 and the actuation-side rod 216 are now movable in the Z1 direction and the Z2 direction in this manner.

The stopper may be provided at actuation unit 201, instead of at the treatment tool unit 200. The angle to be determined as the predetermined angle is as described above. The stopper may be provided at both of the treatment tool unit 200 and the actuation unit 201.

In releasing the treatment tool unit 200 from the state of being attached to the actuation unit 201, the outer shell 425 and the outer shell 421 function as a decoupling mechanism. Specifically, a worker first sets the posture of the distal end portion 11 to the standard posture.

For example, as illustrated in FIG. 3, the following posture of the distal end portion 11 is defined as a “standard posture,” in which all of the longitudinal direction of the jaw 21, the longitudinal direction of the jaw 22, and the longitudinal direction of the wrist portion 23 coincide with the Z-axis direction. In a state in which the distal end portion 11 is in the standard posture, the distal end-side coupling portion 428 of the distal end-side rod 213 and the actuation-side coupling portion 431 of the actuation-side rod 216 are respectively positioned between the distal end-side movement restricting portion 430 of the treatment tool unit 200 and the actuation-side movement restricting portion 424 of the actuation unit 201 in the Z-axis direction.

For example, a worker may input a movement instruction to the operating apparatus 2, illustrated in FIG. 1, so as to make the distal end portion 11 be in the standard posture, which allows the worker to move the distal end-side coupling portion 428 and the actuation-side coupling portion 431 so as to arrange between the distal end-side movement restricting portion 430 and the actuation-side movement restricting portion 424 in the Z-axis direction.

Rotating, by the worker, of the outer shell 425 and/or the outer shell 421 in the J2 direction by the predetermined angle allows the distal end-side movement restricting portion 430 and the actuation-side movement restricting portion 424 to be shifted to, and located at, positions on the XY plane where movements of the distal end-side coupling portion 428 and the actuation-side coupling portion 431 in the Z-axis direction are restricted.

Pulling, by the worker, of the treatment tool unit 200 from the actuation unit 201 in this state allows detachment of the treatment tool unit 200 from the actuation unit 201 while maintaining a state in which the treatment tool unit 200 and the actuation unit 201 are ready for the next attachment, that is, in a state in which the movements of the distal end-side coupling portion 428 and the actuation-side coupling portion 431 in the Z-axis direction are restricted.

Note that the worker may change the posture of the distal end portion 11 to the standard posture manually, instead of through an input of the movement instruction to the operating apparatus 2 illustrated in FIG. 1.

In the above-disclosed embodiment, an example has been described in which the distal end-side coupling portion 428 and the actuation-side coupling portion 431 are respectively connected to flexible elongate elements via the distal end-side rod 213 and the actuation-side rod 216, which are rigid elongate elements. However, the distal end-side coupling portion 428 and/or the actuation-side coupling portion 431 may be connected directly to a flexible elongate element. In a case in which the distal end-side coupling portion 428 is connected directly to a flexible elongate element, the cylindrical portion of the distal end-side coupling portion 428 except the pin, i.e., except the projection 429, corresponds to a rigid distal end-side elongate element specified in the appended claims.

The other configurations and operations are the same as those described in the first to third embodiments. Detailed description thereof will not be repeated here.

### Fifth Embodiment

A medical treatment tool 4b according to a fifth embodiment is configured such that the distal end-side shaft 222 and the actuation-side shaft 212 are fitted to each other by engagement of a claw 414 of the actuation-side shaft 212 into a groove formed in the outer periphery of the distal end-side shaft 222.

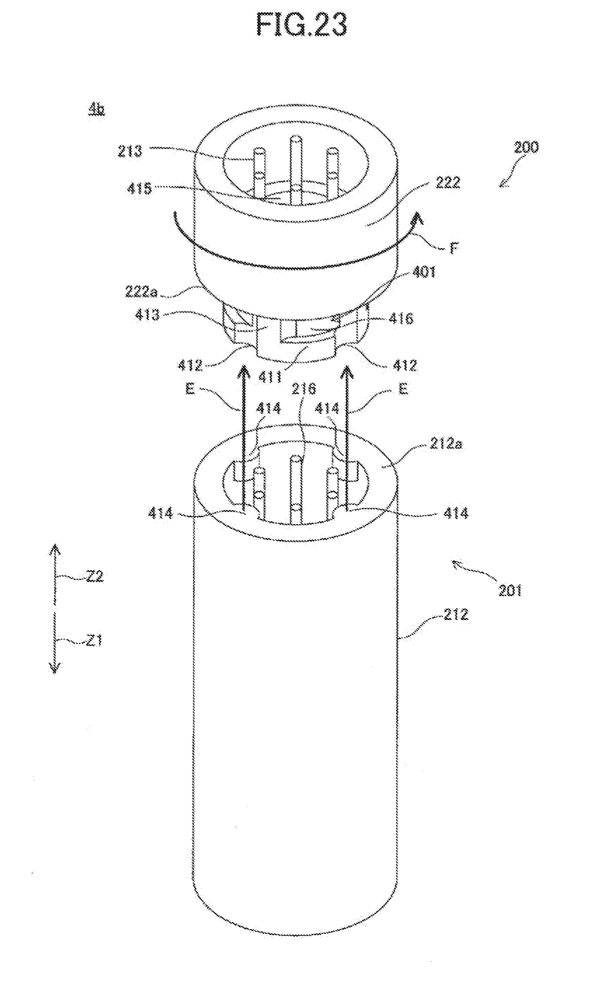

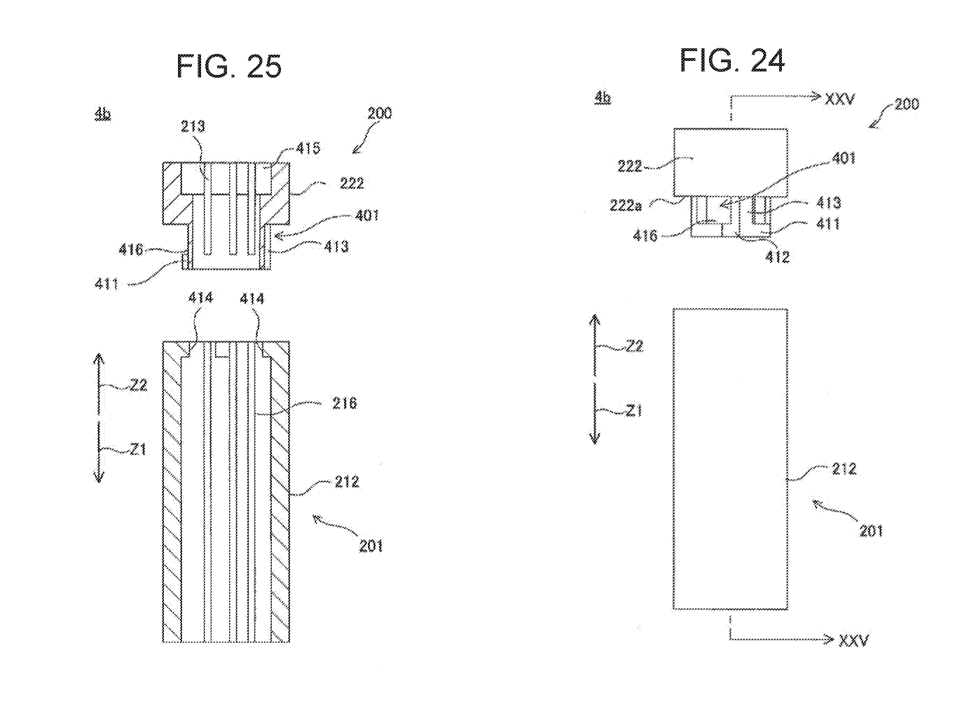

FIG. 23 is a diagram illustrating a perspective view of a configuration (Example 5) of an attachable/detachable mechanism of the medical treatment tool. FIG. 24 is a diagram illustrating a front view of the medical treatment tool in FIG. 23. FIG. 25 is a diagram illustrating a cross-sectional view taken along the line XXV-XXV of FIG. 24. FIGS. 23 to 25 illustrate the distal end-side shaft 222, the actuation-side shaft 212, the distal end-side rod 213, and the actuation-side rod 216 of the medical treatment tool 4b. The distal end-side rod 213 and the distal end portion 11 are coupled to each other with a flexible elongate element, such as a wire, not shown. The actuation-side rod 216 is coupled to an actuation member 106 of the actuation mechanism 15A illustrated in FIG. 8, for example.

Referring to FIGS. 23, 24, and 25, the distal end-side shaft 222 is configured similarly to the distal end-side shaft 222 according to the fourth embodiment, and includes a main body 415, a distal end-side rotatable portion 401 which constitutes an outer shell covering the main body 415 at an end opposite to the distal end, a distal end-side coupling portion (not shown) provided at the distal end-side rod 213, and a distal end-side movement restricting portion (not shown) provided at the distal end-side rotatable portion 401.

The actuation-side rod 216 is configured similarly to the actuation-side rod 216 according to the fourth embodiment, and includes an actuation-side coupling portion (not shown).

The distal end-side rotatable portion 401 is connected to the end 222a of the distal end-side shaft 222 toward the actuation mechanism 15, and has the central axis that coincides with the central axis of the distal end-side shaft 222. The outer diameter of the distal end-side rotatable portion 401 is smaller than the inner diameter of the actuation-side shaft 212.

The distal end-side shaft 222 further includes a rim 411 and a lock 413. The rim 411 rotates around the distal end-side rotatable portion 401 at the end, toward the actuation mechanism 15, of the distal end-side rotatable portion 401, and protrudes in a radial direction of the distal end-side rotatable portion 401. The lock 413 is located between the rim 411 and the end 222a on the outer periphery of the distal end-side rotatable portion 401.

The rim 411 has an indentation 412 at a portion different from a portion where the lock 413 is provided. The indentation 412 includes, for example, four indentations formed at equal intervals. The lock 413 includes, for example, four locks formed at equal intervals. The outer wall of the distal end-side rotatable portion 401, the rim 411, and the four locks 413 constitute four grooves 416.

The actuation-side shaft 212 has a claw 414 provided at the end 212a toward the distal end portion 11. The claw 414 projects toward the central axis of the actuation-side shaft 212, and includes, for example, four claws formed at equal intervals.

In order to attach the distal end-side rod 213 to the actuation-side rod 216, a worker brings the four claws 414 of the actuation-side shaft 212 into the four grooves 416 through the four indentations 412 of the distal end-side shaft 222, as indicated by the arrow E in FIG. 23. Since the movements of the six distal end-side rods 213 in the Z2 direction are inhibited by distal end-side movement restricting portion, the distal end-side coupling portion of each of the six distal end-side rods 213 is coupled to the actuation-side coupling portion of a corresponding one of the six actuation-side rods 216.

In this state, the worker rotates the distal end-side shaft 222 in the direction of arrow F in FIG. 23 such that the four claws 414 moves in the outer circumferential direction of the distal end-side rotatable portion 401 along the four grooves 416, until the four claws 414 comes into contact with the four locks 413. In conjunction with the rotation of the distal end-side rotatable portion 401 of the distal end-side shaft 222 in the F direction, the rim 411 and the lock 413 also rotate in the F direction.

In the above-disclosed configuration, the four claws 414 are engaged with the grooves 416 formed at the outer periphery of the distal end-side rotatable portion 401 of the distal end-side shaft 222, allowing the distal end-side shaft 222 and the actuation-side shaft 212 to be easily fitted to each other.

The other configurations and operations are the same as those described in the first to fourth embodiments. Detailed description thereof will not be repeated here.

Note that the attachable/detachable mechanisms described in the first to fifth embodiments are also applicable to a case where the treatment tool unit 200 does not include a flexible elongate element, such as a wire 14. In this case, the distal end portion 11 is connected directly to the distal end-side rod 213, and is operated in conjunction with the actuation of the distal end-side rod 213. In the above-disclosed configuration, a step of, e.g., adjusting the tension of the wire 14 is not necessary in a process of, e.g., assembling the medical treatment tool 4b. The above-disclosed configuration may further simplify the process of, e.g., assembling the medical treatment tool 4b.

One or more embodiments disclosed herein are meant to be illustrative in all respects and should not be construed to be limiting in any manner. The scope of one or more embodiments is defined not by the above description, but by the scope of claims, and intended to include all modifications within equivalent meaning and scope to those of the claims.

What is claimed is:

1. A treatment tool unit attachable to an actuation unit including an actuation-side shaft which accommodates rigid actuation-side elongate elements, the treatment tool unit comprising:
a distal end portion;
flexible elongate elements coupled to the distal end portion to manipulate the distal end portion;
rigid distal end-side elongate elements each including a first end coupled to a corresponding one of the flexible elongate elements and a second end capable of being coupled to a corresponding one of the actuation-side elongate elements;
a distal end-side shaft which accommodates the rigid distal end-side elongate elements and is attachable to the actuation-side shaft; and
a distal end-side movement restricting portion which restricts movements of the distal end-side elongate elements with respect to the distal end-side shaft toward the distal end portion in an axial direction of the distal end-side shaft when the treatment tool unit is attached to the actuation unit.

2. The treatment tool unit of claim 1, further comprising:
a decoupling mechanism which releases the treatment tool unit from a state of being attached to the actuation unit.

3. The treatment tool unit of claim 1, wherein
each of the distal end-side elongate elements includes a thick portion, and
the distal end-side movement restricting portion is a passage which restricts a range of movement of the thick portion in the axial direction of the distal end-side shaft.

4. The treatment tool unit of claim 2, wherein
the decoupling mechanism includes a rod-shaped engagement member connected to the distal end-side shaft via a connecting portion and capable of being engaged with the actuation-side shaft, and
the engagement member is pivotable about an axis of the connecting portion, the axis being orthogonal to an axis of the distal end-side shaft.

5. The treatment tool unit of claim 2, wherein
the decoupling mechanism includes a force-applying portion which constitutes a portion of an outer periphery of the distal end-side shaft and which is movable toward an inside of the distal end-side shaft.

6. The treatment tool unit of claim 5, wherein
the distal end-side movement restricting portion is caused to function by the force-applying portion.

7. The treatment tool unit of claim 2, wherein
the distal end-side shaft includes a main body and an outer shell,
the decoupling mechanism includes a distal end-side rotatable portion which is rotatable with respect to the main body, and
the distal end-side rotatable portion is the outer shell.

8. The treatment tool unit of claim 7, wherein
the distal end-side movement restricting portion comprises a distal end-side projection which protrudes from the distal end-side shaft toward an inside of the distal end-side shaft.

9. The treatment tool unit of claim 1, wherein
the first end of each of the distal end-side elongate elements is tapered.

10. The treatment tool unit of claim 1, wherein
the distal end-side shaft includes an opening at an end opposite to an end toward the distal end portion, and
a diameter of the opening is larger than a diameter of the other portion of the distal end-side shaft except for the opening.

11. A medical treatment tool, comprising:
the treatment tool unit of claim 1; and
the actuation unit including the actuation-side shaft which accommodates the rigid actuation-side elongate elements, wherein the treatment tool unit is attachable to the actuation unit by coupling the distal end-side shaft with the actuation-side shaft and coupling the rigid distal end-side elongate elements with the actuation-side elongate elements respectively.

12. The medical treatment tool of claim 11, wherein
each of the distal end-side elongate elements includes a pin at the second end thereof, and
each of the actuation-side elongate elements includes a connector to receive the pin, the connector being provided at a side toward the distal end portion.

13. The medical treatment tool of claim 11, further comprising:
a decoupling mechanism which releases the treatment tool unit from a state of being attached to the actuation unit, wherein
the decoupling mechanism includes a force-applying portion which constitutes a portion of an outer periphery of the distal end-side shaft and which is movable toward an inside of the distal end-side shaft,
each of the distal end-side elongate elements includes a hook-shaped distal end-side coupling portion at the second end thereof, and
each of the actuation-side elongate elements includes a hook-shaped actuation-side coupling portion capable of being coupled to the distal end-side coupling portion, the actuation-side coupling portion being provided at a side toward the distal end portion.

14. The medical treatment tool of claim 11, further comprising:
a decoupling mechanism which releases the treatment tool unit from a state of being attached to the actuation unit, wherein
each of the distal end-side shaft and the actuation-side shaft has a main body and an outer shell,
the decoupling mechanism has a distal end-side rotatable portion rotatable with respect to the main body of the distal end-side shaft, and an actuation-side rotatable portion rotatable with respect to the main body of the actuation-side shaft,
the actuation-side rotatable portion is the outer shell of the actuation-side shaft,
the distal end-side rotatable portion is the outer shell of the distal end-side shaft, the distal end-side rotatable portion being engaged with the actuation-side rotatable portion through recess-projection engagement and being rotated in conjunction with the actuation-side rotatable portion.

15. The medical treatment tool of claim 14, wherein
the distal end-side rotatable portion is configured so as not to rotate more than a predetermined angle when the distal end-side rotatable portion rotates in conjunction with the actuation-side rotatable portion, and
the predetermined angle is 360°×1/n×½, where n is the number of the distal end-side elongate elements.

16. The medical treatment tool of claim 11, wherein
the actuation unit includes
a first gear which rotates about a first axis,
a second gear which rotates about a second axis extending in a direction perpendicular to the first axis, and
a third gear which rotates about a third axis extending in a direction perpendicular to the first axis,
each of the actuation-side elongate element includes
a first elongate element which engages with the second gear, and
a second elongate element which engages with the third gear, the second gear and the third gear engage with the first gear, and
the distal end portion is manipulated by a movement of the first elongate element along a longitudinal direction of the medical treatment tool and a movement of the second elongate element in a direction opposite to the movement of the first elongate element, both of the movements occurring in conjunction with a rotation of the first gear.

17. The medical treatment tool of claim 16, wherein
the first gear, the second gear, and the third gear are bevel gears.

18. The medical treatment tool of claim 16, wherein
the first gear is coupled to a receiving member engageable to a transmission member actuated by an actuator.

19. The medical treatment tool of claim 16, wherein
the actuation unit includes three sets of the first gear, the second gear, and the third gear.

20. A surgical system, comprising:
the medical treatment tool of claim 18; and
a manipulator including the transmission member which engages with the receiving member.
